(19) United States
(12) Patent Application Publication
(10) Pub. No.: US 2026/0283969 A1
(43) Pub. Date: Sep. 24, 2026

(54) PROTECTING A BIOACTIVE AND/OR PRECURSOR THEREOF

(71) Applicant: COMMONWEALTH SCIENTIFIC AND INDUSTRIAL RESEARCH ORGANISATION, Acton (AU)

(72) Inventors: Mary Ann AUGUSTIN, Wheelers Hill (AU); Luz SANGUANSRI, Tarniet (AU)

(21) Appl. No.: 19/687,996

(22) Filed: May 26, 2026

Related U.S. Application Data
(63) Continuation of application No. 15/733,458, filed on Feb. 1, 2019, now abandoned.

(30) Foreign Application Priority Data
Feb. 2, 2018 (AU) 2018900326

(51) Int. Cl. A61K 9/50; A23B 20/10; A23D 7/005; A23D 7/01; A23D 7/02; A23K 20/147; A23K 20/158; A23K 20/163; A23K 50/80; A23L 19/00; A23L 33/12; A23L 33/125; A23L 33/17; A23P 10/28; A23P 10/35; A61K 8/02; A61K 8/04; A61K 8/06; A61K 8/11; A61K 8/36; A61K 8/46; A61K 8/49; A61K 8/60; A61K 8/92; A61K 8/9789; A61K 8/9794; A61K 9/00; A61K 9/107; A61K 9/19; A61K 9/20; A61K 31/00; A61K 31/105; A61K 31/202; A61K 31/26; A61K 31/352; A61K 31/353; A61K 31/7028; A61K 35/60; A61K 36/31; A61K 36/88

(52) U.S. Cl. CPC A61K 9/5089; A23B 20/10; A23D 7/0053; A23D 7/011; A23D 7/02; A23K 20/147; A23K 20/158; A23K 20/163; A23K 50/80; A23L 19/09; A23L 33/12; A23L 33/125; A23L 33/17; A23P 10/28; A23P 10/35; A61K 8/022; A61K 8/044; A61K 8/06; A61K 8/11; A61K 8/361; A61K 8/46; A61K 8/498; A61K 8/602; A61K 8/925; A61K 8/9789; A61K 8/9794; A61K 9/0014; A61K 9/0029; A61K 9/0053; A61K 9/107; A61K 9/19; A61K 9/2081; A61K 9/2095; A61K 9/5036; A61K 9/5052; A61K 9/5063; A61K 31/105; A61K 31/202; A61K 31/26; A61K 31/352; A61K 31/353; A61K 31/658; A61K 31/7028; A61K 35/60; A61K 36/31; A61K 36/88; A23V 2002/00; A61K 2236/15; A61K 2236/17; A61K 2236/19

(57) ABSTRACT

The invention relates to a method of producing an emulsion or suspension from a biomass. The invention also relates to producing a powder or products produced therefrom. The invention also relates to an emulsion or suspension produced by a method as described herein. The invention also relates to a powder produced by a method as described herein or products produced therefrom.

### CROSS-REFERENCE TO RELATED APPLICATIONS

This application is a continuation of U.S. patent application Ser. No. 15/733,458, filed Jul. 31, 2020, which is a national phase application under 35 U.S.C. § 371 of International Application No. PCT/AU2019/050079, filed Feb. 1, 2019, which claims the benefit of priority of Australian Patent Application No. 2018900326, filed Feb. 2, 2018; all of which are hereby incorporated by reference in their entirety.

### FIELD OF THE INVENTION

The invention relates to a method of producing an emulsion or suspension from a biomass. The invention also relates to producing a powder or products produced therefrom. The invention also relates to an emulsion or suspension produced by a method as described herein. The invention also relates to a powder produced by a method as described herein or products produced therefrom.

### BACKGROUND OF THE INVENTION

Bioactives, such as oxygen sensitive fatty acids and oils, are desirable ingredients for foods, supplements and/or cosmetics. However, because many oils and bioactive molecules are susceptible to oxidation and degradation reactions when exposed to various environments (such as oxygen, heat, pH, or enzymes) these ingredients can degrade before ingestion or use, cannot be stored in a form suitable for ingestion or use, or do not get to the desired site in the body after ingestion. Oxygen sensitive oils include those containing polyunsaturated fatty acids such as omega-3, omega-6, or omega-9 fatty acids. Unstable bioactive ingredients include components which are water soluble (such as polyphenols which are unstable at high pH) or oil soluble (such as carotene which is sensitive to oxygen) or sparingly soluble in oil or water (such as resveratrol, curcumin). 58.

Encapsulation has been used for protection and delivery of lipophilic and hydrophilic bioactives but there are still challenges in delivery and choosing the most appropriate encapsulation system for delivery (Augustin and Sanguansri, 2015; McClements, 2015). Because of the health promoting properties of omega-3 oils, there has been significant interest in stabilisation of these oils as they are very prone to oxidation (Sanguansri and Augustin, 2006; Drusch and Manino, 2007).

Methods of encapsulating oxygen sensitive oils and bioactives are known, however, these methods require purified or substantially purified protein (such as isolated whey protein, isolated soy protein, or casein and carbohydrate) which are not economical for many products.

Purified proteins and purified carbohydrates may be used alone or in combination as encapsulating matrices for the delivery of bioactives (Augustin and Hemar, 2009; Aditya et al., 2017). For example, starches have commonly been used as the wall material for encapsulation (Hoyos-Leyva et al., 2018) and proteins have been found to be useful for delivery because of the many desirable functional properties that lend themselves to encapsulation (Subirade and Chen, 2008; Livney, 2010). MicroMAX® microencapsulation technology uses purified protein (preferably casein) and purified carbohydrates to produce an encapsulant for oils. Heated protein-carbohydrate blends (MicroMAX® microencapsulation technology) were found to be superior to corresponding physical blends of proteins and carbohydrates (WO 01/74175; Augustin et al., 2006). Purified proteins and purified carbohydrates used in such processes are selected to be colourless and lacking flavours and can be costly due to the purification steps involved in their isolation.

Thus, there is a requirement for new formulations and processes to produce products comprising oxygen sensitive bioactives, such as fatty acids and oils, that are prone to degradation during storage, processing and in the gastrointestinal tract.

### SUMMARY OF THE INVENTION

The present inventors have developed a method of producing an emulsion, suspension, or powder comprising protein and carbohydrate obtained from a single source.

In an aspect, the present invention provides a method of producing an emulsion or suspension, the method comprising:
i) obtaining an aqueous mixture comprising protein and carbohydrate from a biomass of a single species of organism;
ii) optionally adding oil to the aqueous mixture; and
iii) forming an emulsion or suspension comprising a bioactive and/or bioactive precursor.

In an embodiment, the aqueous mixture is an aqueous suspension.

In an aspect, the present invention provides a method of producing a powder comprising an entrapped or encapsulated bioactive and/or bioactive precursor, the method comprising:
i) obtaining an aqueous mixture comprising protein and carbohydrate from a biomass of a single species of organism;
ii) adding oil to the aqueous mixture;
iii) forming an emulsion or suspension comprising the bioactive and/or bioactive precursor; and
iv) forming a powder comprising an entrapped or encapsulated bioactive and/or bioactive precursor from the emulsion or suspension.

In an embodiment, the present invention provides a method as described herein, wherein the bioactive and/or bioactive precursor is one or more of:
i) a component of the biomass;
ii) the oil or a component thereof in step ii);
iii) a component added to the oil before the oil is added to the aqueous mixture in step ii);
iv) a component infused in the oil before or during step ii);
v) a component of the further biomass; and
vi) is a component added in step i), ii) and iii) of the method.

In an embodiment, the bioactive is i) and ii).

In an embodiment, the bioactive precursor is i).

In an embodiment, the bioactive is formed in or after step i) or in step ii).

In an aspect, the present invention provides a matrix comprising protein and carbohydrate from a biomass of a single species of organism.

In an aspect, the present invention provides a bioactive and/or bioactive precursor entrapped or encapsulated in a matrix comprising protein and carbohydrate of a biomass from a single species of organism wherein the entrapped or encapsulated bioactive and/or bioactive precursor is resistant to oxygen degradation when compared to the bioactive and/or bioactive precursor before entrapment or encapsulation.

In an aspect, the present invention provides an emulsion or suspension produced by the method as described herein.

In an aspect, the present invention provides an emulsion or suspension produced by the method as described herein.

In an aspect, the present invention provides a powder comprising an entrapped or encapsulated bioactive and/or bioactive precursor and comprising protein and carbohydrate from a single species of organism.

In an aspect, the present invention provides a powder, produced by the method as described herein.

In an aspect, the present invention provides a product comprising the emulsion or suspension produced by the method as described herein, the matrix as described herein, the bioactive and/or bioactive precursor entrapped or encapsulated in a matrix as described herein, or the emulsion or suspension as described herein.

In an aspect, the present invention provides a product comprising the powder produced by the method described herein, or the powder described herein.

The present inventors have also surprisingly found that lipid based compositions improve the stability of isothiocyanates (such as sulforaphane) and/or an isothiocyanate precursors (such as glucosinolate). Thus, in a further aspect, the present invention provides a pharmaceutical or cosmetic composition, which comprises an isothiocyanate and/or an isothiocyanate precursor, a lipid, and a pharmaceutical and/or cosmetic excipient.

In an aspect, the present invention provides a method of producing an emulsion comprising an isothiocyanate or isothiocyanate precursor, the method comprising:

providing a mixture comprising water, a lipid, and an isothiocyanate or isothiocyanate precursor, thereby forming an emulsion.

In an aspect, the present invention provides an emulsion comprising water, a lipid, and an isothiocyanate and/or isothiocyanate precursor.

In an aspect, the present invention provides a method of preparing a powder comprising an isothiocyanate and/or isothiocyanate precursor, comprising: preparing an emulsion as described herein, and subjecting the emulsion to drying conditions, thereby removing water and forming a powder.

In an aspect, the present invention provides a method of preparing a pharmaceutical or cosmetic composition, comprising: preparing an emulsion as described herein, or preparing a powder as described herein, and converting the emulsion or dried composition to a pharmaceutical or cosmetic composition.

In an aspect, the present invention provides a method of therapy or prophylaxis of a condition, comprising administering to a subject in need thereof an effective amount of a pharmaceutical composition, emulsion or powder as described herein.

In an aspect, the present invention provides a pharmaceutical composition, emulsion or powder as described herein, for use in therapy or prophylaxis of a condition.

In an aspect, the present invention provides a method of treating or preventing a condition in a subject, comprising administering to the subject an effective amount of a pharmaceutical composition, emulsion or powder as described herein.

In an aspect, the present invention provides use of the emulsion as described herein, or the powder as described herein in the manufacture of a medicament for the treatment of a condition.

In an aspect, the present invention provides the method or use as described herein, wherein the condition is selected from: cancer, diabetes, cardiovascular, autism, osteoporosis, neuroprotective diseases, inflammation, oxidative stress and gut health.

Any embodiment herein shall be taken to apply mutatis mutandis to any other embodiment unless specifically stated otherwise. For instance, as the skilled person would understand examples of bioactives and/or bioactive precursors for the above methods of the invention equally apply to emulsions, suspensions, powders and products of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1 Shows the physical stability of oil-in-water emulsion using broccoli containing protein and carbohydrates as an encapsulant compared to oil and water dispersion at A) time 0 mins and B) time 120 min after creation of the emulsion. This Figure also shows the preparation of the aqueous phase suspension using freeze dried broccoli powder, adding sufficient amount of water until a flowable mixture (7.46% TS) in achieved prior to its use as encapsulant. A) Shows 14.29% total solids (TS), B) shows 10.64% TS, C) shows 8.48% TS and D) shows 7.46% TS.

FIG. 2 Shows the preparation of the aqueous phase suspension using raw broccoli, by adding the required amount of water until a flowable mixture is achieved prior to its use as encapsulant. A) 7.66% TS, B) 6.87% TS, C) 6.23% TS, and D) 4.99% TS.

FIG. 3 Shows the emulsions containing omega-3 oil A) after preparation, B) after overnight storage and C) after freeze drying. F1 and F2 using broccoli as encapsulant, C1 using heated casein-carbohydrate as encapsulant, and C2 using Tween as emulsifier.

FIG. 4 Oxipres test results showing the induction period (IP)/uptake of oxygen of samples [Emulsions from FIG. 3A (9.5% TS, 4.8% oil)] tested at 80° C. with initial 5 bar oxygen pressure. Sample tested was 83 g emulsion (4 g matrix solids and 4 g oil in sample). The IP (h), when there is a significant change in oxygen consumption, is only observed for the sample with Tween as emulsifier and that with heated casein-carbohydrate as encapsulation. Samples using broccoli as encapsulant do not have a distinctive IP up to 20 hr, when the test was stopped. The slow oxygen uptake in these samples is in part due to the oxygen uptake by the broccoli matrix. Oxipres for neat tuna oil (see FIG. 6) is 9 hrs.

FIG. 5 Oxipres test results showing the IP/uptake of oxygen of samples [Freeze dried powder from FIG. 3C (50% tuna oil)] tested at 80° C. with initial 5 bar oxygen pressure. Sample tested was 8 g powder (4 g matrix solids and 4 g oil in sample). Samples using broccoli as encapsulant do not have a distinctive IP up to 43 hrs, when the test was stopped. The slow oxygen uptake in these samples is in part due to the oxygen uptake by the broccoli matrix. The IP for neat tuna oil from Oxipres data (see FIG. 6) is 9 hrs.

FIG. 6 Oxipres test results showing the IP/uptake of oxygen of tuna oil, canola oil and high-DHA canola oil tested at 80° C. with initial 5 bar oxygen pressure. A clear IP is observed for each oil.

FIG. 7 Oxipres test on broccoli matrix (without oil) showing the effect of different amounts of vegetable matrix on oxygen uptake.

FIG. 8 Oxipres test results of freeze dried omega-3 broccoli powder (12.5% tuna oil or canola oil), tested at 80° C. with initial 5 bar oxygen pressure. The total solids of emulsion before drying was 5.7%. Sample tested was 20 g powder (17.5 g matrix and 2.5 g oil). The slow oxygen uptake in these samples is in part due to the oxygen uptake by the broccoli matrix.

FIG. 9 Oxipres test results of freeze dried omega-3 broccoli powder (25% tuna oil or DHA canola oil), tested at 80° C. with initial 5 bar oxygen pressure. The total solids of emulsion before drying was 6.6%. Sample tested was 10 g powder (7.5 g matrix and 2.5 g oil). The slow oxygen uptake in these samples is in part due to the oxygen uptake by the broccoli matrix. The IP (h) is where there is a significant increase in oxygen uptake (sharp decline in oxygen pressure).

FIG. 10 Oxipres test results of freeze dried omega-3 broccoli powder (50% tuna oil or DHA canola oil), tested at 80° C. with initial 5 bar oxygen pressure. The total solids of emulsion before drying was 9.5%. Sample tested was 5 g powder (2.5 g matrix and 2.5 g oil). The slow oxygen uptake in these samples is in part due to the oxygen uptake by the broccoli matrix.

FIG. 11 Oxipres test results showing the oxygen uptake of omega-3 broccoli emulsion samples tested at 80° C. with initial 5 bar oxygen pressure. Emulsions were prepared with two heat-treatment (75° C.-2 min and 100° C.-30 min) at 4% aqueous solids (3.8% oil and 7.7% total solids) and 6% aqueous solids (5.7% oil and 11.3% total solids). Sample tested contained 4 g oil and 4 g matrix. Emulsions using broccoli as encapsulant do not have a distinctive IP up to 42 hrs. The slow oxygen uptake in these samples is in part due to the oxygen uptake by the broccoli matrix.

FIG. 12 Oxipres test results showing the IP of freeze dried omega-3 broccoli powder (50% tuna oil), tested at 80° C. with initial 5 bar oxygen pressure. Samples were prepared with two heat-treatment (75° C.-2 min and 100° C.-30 min) at 5% and 6% aqueous solids (5.7% oil and 11.3% total solids). Sample tested contained 4 g oil and 4 g matrix.

FIG. 13 Oxipres test results showing the IP of freeze dried omega-3 broccoli powders (50% tuna oil), tested at 80° C. with initial 5 bar oxygen pressure. The broccoli encapsulant was subjected to two heat-treatment (75° C.-2 min and 100° C.-30 min) and used as is (no drying “fresh broccoli”) or reconstituted from “freeze dried broccoli” powder. The IP (h) is where there is a significant increase in oxygen uptake (sharp decline in oxygen pressure).

FIG. 14 Oxipres test results showing the oxygen uptake of omega-3 broccoli emulsion samples tested at 80° C. with initial 5 bar oxygen pressure. The broccoli encapsulant was used at different stages of processing and made up to 5% aqueous solids. The emulsions were prepared at 9.5% TS and 4.8% oil. The IP (h) is where there is a significant increase in oxygen uptake (sharp decline in oxygen pressure). Sample tested contained 4 g oil and 4 g matrix. The slow oxygen uptake in these samples is in part due to the oxygen uptake by the broccoli matrix.

FIG. 15 Oxipres test results showing the IP of freeze dried omega-3 carrot powder (50% tuna oil), tested at 80° C. with initial 5 bar oxygen pressure. Results showing two heat-treatment (75° C.-2 min and 100° C.-30 min) used. The total solids of emulsion before drying was 9.5%. Sample tested was 8 g powder (4 g matrix and 4 g oil). There is no clear IP. A sudden increase in pressure is shown leading to release of volatiles (marked IP), therefore not possible to obtain rate of oxygen uptake beyond IP. The IP (h) is where there is a significant increase in oxygen uptake (sharp decline in oxygen pressure). Sharp peak is evidence of an interaction which led to a marked increase in pressure.

FIG. 16 Oxipres test results showing the IP of omega-3 carrot powders (50% tuna oil) using “fermented” and “non-fermented” carrot as encapsulant for omega-3 oils, tested at 80° C. with initial 5 bar oxygen pressure. The total solids of emulsion before drying was 9.5%. Sample tested was 8 g powder (4 g matrix and 4 g oil). A sudden increase in pressure is shown leading to release of volatiles (marked IP), therefore not possible to obtain rate of oxygen uptake beyond IP. Sharp peak is evidence of an interaction which led to a marked increase in pressure. There is no clear IP for non-fermented carrot as encapsulant. The IP (h) is where there is a significant increase in oxygen uptake (sharp decline in oxygen pressure).

FIG. 17 Oxipres test results showing the IP of freeze dried omega-3 tomato powder (50% tuna oil), tested at 80° C. with initial 5 bar oxygen pressure. There is no clear IP. A sudden increase in pressure is shown leading to release of volatiles (marked IP), therefore not possible to obtain rate of oxygen uptake beyond IP. The IP (h) is where there is a significant increase in oxygen uptake (sharp decline in oxygen pressure). Sharp peak is evidence of an interaction which led to a marked increase in pressure. Results showing two heat-treatment (75° C.-2 min and 100° C.-30 min) used. The total solids of emulsion before drying was 9.5%. Sample tested was 8 g powder (4 g matrix and 4 g oil). Increase temperature-time treatment for tomato show longer IP (better protection from oxidation of omega-3 oil).

FIG. 18 Oxipres test results showing the IP of freeze dried omega-3 mushroom powders (25% and 50% oil), tested at 80° C. with initial 5 bar oxygen pressure. There is no clear IP (h) where there is a significant increase in oxygen uptake (sharp decline in oxygen pressure). Results showing two heat-treatment of mushroom as encapsulant (75° C.-2 min and 100° C.-30 min) for 50% oil powder. The total solids of emulsion before drying was 9.5%. Sample tested was 8 g powder (4 g matrix and 4 g oil) for 50% oil powder and 12 g powder (9 g matrix and 3 g oil) for 25% oil powder. Lower oil loading (25% oil) show longer IP (better protection from oxidation of omega-3 oil) compared to 50% oil powder.

FIG. 19 Oxipres test results showing the IP of freeze dried omega-3 cauliflower powders (25% and 50% oil), tested at 80° C. with initial 5 bar oxygen pressure. IP observed for 50% oil powder, but no clear IP for 25% oil powder, therefore not possible to obtain rate of oxygen uptake beyond IP. The IP (h) is where there is a significant increase in oxygen uptake (sharp decline in oxygen pressure). Results showing heat-treatment of cauliflower as encapsulant at (75° C.-2 min), and two oil loading (50% and 25% oil). The total solids of emulsion before drying was 9.5%. Sample tested was 8 g powder (4 g matrix and 4 g oil) for 50% oil powder and 12 g powder (9 g matrix and 3 g oil) for 25% oil powder.

FIG. 20 Oxipres test results showing the IP of freeze dried omega-3 kale powders (25% and 50% oil), tested at 80° C. with initial 5 bar oxygen pressure. IP observed for 25% oil powder, but no clear IP for 50% oil powder, therefore not possible to obtain rate of oxygen uptake beyond IP. The IP (h) is where there is a significant increase in oxygen uptake (sharp decline in oxygen pressure). Sharp peak for 50% oil powder is evidence of an interaction which led to a marked increase in pressure observed for 50% tuna oil powder. Results showing heat-treatment of kale as encapsulant at (75° C.-2 min), and two oil loading (50% and 25% oil). The total solids of emulsion before drying was 9.5%. Sample tested was 8 g powder (4 g matrix and 4 g oil) for 50% oil powder and 12 g powder (9 g matrix and 3 g oil) for 25% oil powder.

FIG. 21 Oxipres test results showing the IP of freeze dried omega-3 brussel sprouts powders (25% and 50% oil), tested at 80° C. with initial 5 bar oxygen pressure. IP observed for 50% oil powder, but no clear IP for 25% oil powder, therefore not possible to obtain rate of oxygen uptake beyond IP. The IP (h) is where there is a significant increase in oxygen uptake (sharp decline in oxygen pressure). Results showing heat-treatment of brussel sprouts as encapsulant at (75° C.-2 min), and two oil loading (50% and 25% oil). The total solids of emulsion before drying was 9.5%. Sample tested was 8 g powder (4 g matrix and 4 g oil) for 50% oil powder and 12 g powder (9 g matrix and 3 g oil) for 25% oil powder.

FIG. 22 Oxipres test results showing the IP of freeze dried omega-3 snow pea powders (25% and 50% oil), tested at 80° C. with initial 5 bar oxygen pressure. No clear IP, therefore not possible to obtain rate of oxygen uptake beyond IP. The IP (h) is where there is a significant increase in oxygen uptake (sharp decline in oxygen pressure). Sharp peak for 50% oil powder is evidence of an interaction which led to a marked increase in pressure observed for 50% tuna oil powder. Results showing heat-treatment of snow peas as encapsulant at (75° C.-2 min), and two oil loading (50% and 25% oil). The total solids of emulsion before drying was 9.5%. Sample tested was 8 g powder (4 g matrix and 4 g oil) for 50% oil powder and 12 g powder (9 g matrix and 3 g oil) for 25% oil powder.

FIG. 23 Oxipres test results showing the IP of freeze dried omega-3 garlic powders (25% and 50% oil), tested at 80° C. with initial 5 bar oxygen pressure. IP observed for 25% oil powder, but no clear IP for 50% oil powder, therefore not possible to obtain rate of oxygen uptake beyond IP. The IP (h) is where there is a significant increase in oxygen uptake (sharp decline in oxygen pressure). Sharp peak for 50% oil powder is evidence of an interaction which led to a marked increase in pressure observed for 50% tuna oil powder. Results showing heat-treatment of garlic as encapsulant at (75° C.-2 min), and two oil loading (50% and 25% oil). The total solids of emulsion before drying was 9.5%. Sample tested was 8 g powder (4 g matrix and 4 g oil) for 50% oil powder and 12 g powder (9 g matrix and 3 g oil) for 25% oil powder.

FIG. 24 Oxipres test results of freeze dried omega-3 carrot powders (25% tuna oil) with added plant protein (pea protein, soy protein (SPI)) or dairy protein, Na-caseinate), tested at 80° C. with initial 5 bar oxygen pressure. The total solids of emulsion before drying was 9.5% (2.4% oil). The IP (h) is where there is a significant increase in oxygen uptake (sharp decline in oxygen pressure). Sample tested was 10 g powder (2.5 g oil and 7.5 g matrix). A sudden increase in pressure is shown leading to release of volatiles, therefore not possible to obtain IP and rate of oxygen uptake, except for carrot with pea protein added as encapsulant. Sharp peak is evidence of an interaction which led to a marked increase in pressure. The IP (h) is where there is a significant increase in oxygen uptake (sharp decline in oxygen pressure).

FIG. 25: Oxipres test results showing the IP of spray dried (25% tuna oil) omega-3 matcha powder with different protein: carbohydrate ratio, tested at 80° C. with initial 5 bar oxygen pressure compared to that of neat tuna oil. The matcha powder was reconstituted from a commercial sample of matcha (green tea) powder. IP observed for tuna oil only and powders encapsulated with 1:3 and 1:4 protein to carbohydrate ratios but no clear IP for powders encapsulated with 1:2 and 8:9 protein to carbohydrate ratios and therefore not possible to obtain rate of oxygen uptake beyond IP. Encapsulant containing 8:9 protein to carbohydrate ratio was that of matcha powder only.

FIG. 26 Oxipres test results showing the IP of spray dried (50% tuna oil) omega-3 broccoli powder, tested at 80° C. with initial 5 bar oxygen pressure compared to that of 50% tuna oil powder using heated casein-carbohydrate as encapsulant. The broccoli encapsulant was reconstituted from “freeze dried broccoli” powder.

FIG. 27 Oxipres test results showing the IP of freeze dried (50% tuna oil) omega-3 oil powders using broccoli puree and fermented broccoli puree as encapsulants, tested at 80° C. with initial 5 bar oxygen pressure.

FIG. 28: Quantitative analysis of secondary lipid oxidation in freeze dried powders (50% tuna oil) encapsulated in different vegetable matrices after storage at 40° C. for 4 weeks. Vegetable encapsulants (left to right: Broccoli, Carrot, Fermented Carrot, Tomato, Mushroom, Cauliflower, Kale, Brussel sprouts, Snow peas, Garlic).

FIG. 29: Oxipres test results showing the induction period (IP) of extruded broccoli snacks (10% Hi-DHA tuna oil) and tabletted omega-3 broccoli formulation (25% Hi-DHA tuna oil) tested at 80° C. with initial 5 bar oxygen pressure. There is no clear IP (h) for the tabletted formulation but a clear IP is shown for extruded formulation. Sample tested was 40 g for the extrudate (36 g matrix and 4 g oil) and 16 g sample tablet formats (16 g excipient and 4 g oil).

FIG. 30: Oxipres test results showing the induction period (IP) of freeze dried omega-3 broccoli powders (50% oil), tested at 80° C. with initial 5 bar oxygen pressure. There is no clear IP (h) where there is a significant increase in oxygen uptake (sharp decline in oxygen pressure). Results showing pre-treatment (using ultrasound or microwave) to the aqueous phase (or post-emulsification treatment (using HPP or microwave). The total solids of aqueous phase is 5%, and total solids of emulsion was 9.5%. Sample tested was 8 g powder (4 g matrix and 4 g oil).

### DETAILED DESCRIPTION

### General Techniques and Definitions

Unless specifically defined otherwise, all technical and scientific terms used herein shall be taken to have the same meaning as commonly understood by one of ordinary skill in the art.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about”, unless stated to the contrary, refers to +/−10%, more preferably +/−5%, even more preferably +/−1%, of the designated value.

As used herein “component” refers to a part or element of a larger whole.

As used herein “protein” or “polypeptide” refers to macromolecules comprising carbon, hydrogen, oxygen, nitrogen and usually sulfur comprising polymers of amino acids linked together by peptide binds.

As used herein “carbohydrate” refers to a class of molecules of the general formula Cx(H2O)y.

As used herein, the term “resistant to oxygen degradation”, or similar phrases, refers to reducing the susceptibility of a bioactive substance, such as a fatty acid, to oxidation. In an embodiment, the susceptibility of the substance to oxidation is reduced by entrapping or encapsulating the substance to reduce exposure to oxygen. In an embodiment, this includes entrapping or encapsulating the substance with molecules with oxygen sequestration ability. Assessment of oxidative resistance may be performed by any method known to a person skilled in the art. For example, the oxidative resistance of a fat and oil may be based on the oxidation of oil with oxygen under pressure. In such a test, the consumption of oxygen, results in a pressure drop during the test which is due to the uptake of oxygen by the sample during oxidation. The oxidation rate is accelerated when carried out at elevated pressure and temperature. In an embodiment, the oxidative resistance is assessed using an Oxipres (e.g. a Mikrolab Aarhus A/S apparatus Højbjerg, Denmark). In an embodiment, an emulsion, suspension and/or powder containing fat or oil (e.g. polyunsaturated oils) is exposed to high temperature and high oxygen pressure. In an embodiment, the oxidative resistance is assessed at 80° C. and 5 bar initial oxygen pressure. In an embodiment, the induction period (IP, h) is determined, which is related to oxidative stability of the samples. A longer IP (h) indicates that the sample is more resistant (more stable in the presence of oxygen) to oxidation during storage. Other methods for measuring lipid oxidation include, for example, peroxide value, para-anisidine value and headspace analysis of volatiles (eg aldehydes such as propanal and EE-2,4-heptadienal which are secondary oxidation products from oxidation of omega-3 fatty acids) and change in % individual unsaturated fatty acids (e.g. EPA and DHA) in stored samples. In an embodiment, oxidation is not necessarily relative to solvent extractable free-fat (i.e. the free fat level is not an indicator of IP or susceptibility of oils to oxidation in powders).

As used herein “temperature degradation”, or similar phrases, refers to degradation of a bioactive (e.g. a fatty acid) or bioactive precursor due to exposure to low or high temperature. In an embodiment, the sensitivity to degradation by temperature is reduced by binding of the bioactive or bioactive precursor to a protein or carbohydrate during the method of producing an emulsion, suspension or powder as described herein.

As used herein “moisture degradation”, or similar phrases, refers to degradation of a bioactive (e.g. a fatty acid) or bioactive precursor due to exposure to low or high moisture. In an embodiment, the sensitivity to degradation by moisture is reduced by binding of the bioactive or bioactive precursor to a protein or carbohydrate during the method of producing an emulsion, suspension or powder as described herein.

As used herein, the term “pH degradation”, or similar phrases, refers to the degradation of a bioactive (e.g. a fatty acid) or bioactive precursor due to exposure to a low or high pH. In an embodiment, low pH is a pH<7. In an embodiment, high pH is a pH>7. In an embodiment, the sensitivity to degradation by pH is reduced by binding of the bioactive or bioactive precursor (e.g. phytonutrients) to a protein or carbohydrate during the method of producing an emulsion, suspension or powder as described herein.

As used herein, the term “light degradation”, or similar phrases, refers to the degradation of a bioactive (e.g. a carotenoid) or bioactive precursor, due to exposure to light. In an embodiment, the sensitivity to light degradation is reduced by binding of the bioactive or bioactive precursor to a protein or carbohydrate during the method of producing an emulsion, suspension or powder as described herein.

As used herein, “entrapment” or “entrapped” or “entrapping” refers to binding or partitioning of a bioactive or bioactive precursor, such as a phytonutrient, to one or more components of an emulsion, suspension or encapsulant matrix as described herein. In an embodiment, the component is carbohydrate or a protein. In an embodiment, entrapment increases the resistance of the bioactive or bioactive precursor to one or more of degradation by oxygen, temperature, pH, moisture and light.

As used herein, “encapsulation” or “encapsulated” refers to forming of a functional barrier around a bioactive or bioactive precursor, such as lipids and lipid soluble components in an emulsion, suspension or encapsulant matrix of, or produced by a method of, the invention. In an embodiment, encapsulation increases the resistance of the bioactive or bioactive precursor to one or more of degradation by oxygen, temperature, pH, moisture and light.

As used herein “species of organism” refers to a subdivision of a genus. In an embodiment, “species of organism” refers to a group of organisms consisting of individuals capable of breeding among themselves.

As used herein, “polyphenol” refers to a compound comprising more than one phenolic hydroxyl group. In an embodiment, the polyphenol is selected from one or more of: anthocyanins, dihydrochalcones, flavan-3-ols, flavanones, flavones, flavonols and isoflavones, curcumin, resveratrol, benzoic acid, phenyl acetic acid, hydroxycinnamic acids, coumarins, napthoquinones, xanthones, stilbenes, chalcones, tannins, phenolic acids, and catechins (e.g. epigallocatechin gallate (EGCg), epigallocatechin (EGC), epicatechin gallate (ECg), epicatechin (EC), and their geometric isomers gallocatechin gallate (GCg), gallocatechin (GC), catechin gallate (Cg) and catechin.

As used herein, “bioactive organo-sulfur containing compound/s” includes sulfur containing compounds such as glucosinolates, isothiocyanates and allium compounds (e.g. aliin, allicin, ajoene, allylpropyl disulfide, diallyl trisulfide, sallylcysteine, vinyldithiines, S-allylmercaptocystein).

### Biomass

The present invention relates, at least in part, to methods for producing an emulsion, suspension, powder, or a product produced therefrom, from biomass comprising protein and carbohydrate from a single (first) species of organism. Thus, the protein and carbohydrate have not been separated from each other before being used in a method of the invention.

Whilst in some embodiments the whole biomass can be used, in other embodiments the biomass has been processed to remove, or reduce the concentration of, one or more components of the biomass. In an embodiment, less than about 50% of the biomass is removed before being used in a method of the invention. In an embodiment, less than about 40% of the biomass is removed before being used in a method of the invention. In an embodiment, less than about 30% of the biomass is removed before being used in a method of the invention. In an embodiment, less than about 20% of the biomass is removed before being used in a method of the invention. In an embodiment, less than about 10% of the biomass is removed before being used in a method of the invention. In an embodiment, less than about 5% of the biomass is removed before being used in a method of the invention. In an embodiment, less than about 1% of the biomass is removed before being used in a method of the invention. In an embodiment, none of the biomass is removed before being used in a method of the invention.

In an embodiment, the biomass is dried or concentrated to remove water. In an embodiment, drying removes about 60% to about 90% of the weight of the biomass. In an embodiment, drying removes about 70% to about 90% of the weight of the biomass. In an embodiment, drying removes about 80% to about 90% of the weight of the biomass.

In an embodiment, the biomass comprises protein and carbohydrate from a single species of organism only (no protein or carbohydrate from a further species of organism). In an embodiment, the biomass further comprises protein and carbohydrate from one or more further species of organism (e.g. second, third, fourth, fifth etc. species of organism). As with the biomass from the first species of organism, the protein and carbohydrate from the further species has not been separated from each other before being used in a method of the invention. In an embodiment, the biomass further comprises protein and carbohydrate from a second species of organism. In an embodiment, the biomass further comprises protein and carbohydrate from a second and a third species of organism. In an embodiment, the biomass further comprises protein and carbohydrate from a second, third and fourth species of organism.

In an embodiment, the biomass and/or further biomass comprises fiber which has not been separated from the protein and carbohydrate of the biomass and/or further biomass.

In an embodiment, the biomass and/or further biomass comprises catechins which has not been separated from the protein and carbohydrate of the biomass and/or further biomass.

In an embodiment, the biomass and/or further biomass has a protein to carbohydrate ratio of between 1:1 and 1:10.5. In an embodiment, the biomass and/or further biomass has a protein to carbohydrate ratio of between about 1:4.5 and 4:1. In an embodiment, the biomass and/or further biomass has a protein to carbohydrate ratio of between about 1:2.5 and 2:1. In an embodiment, the biomass and/or further biomass has a protein to carbohydrate ratio of about 1:2.4. In an embodiment, the biomass and/or further biomass additionally comprises fiber. In an embodiment, the biomass or further biomass has a protein to carbohydrate ratio as shown in Table 1.

In an embodiment, the biomass or further biomass is green tea leaf powder (matcha). In an embodiment, matcha, (~2% moisture) comprises about 35.5% protein, about 39.6% carbohydrate, about 5.9% fat and about 6.0% fat on a dry basis. In an embodiment, matcha comprises about 13.1% catechins.

In an embodiment, protein is added to the biomass and/or further biomass to form a protein to carbohydrate ratio of between about 1:1 and 1:10.5. In an embodiment, protein is added to the biomass and/or further biomass to form a protein to carbohydrate ratio of between about 1:4.5 and 4:1. In an embodiment, protein is added to the biomass and/or further biomass to form a protein to carbohydrate ratio of between about 1:2.5 and 2:1.

TABLE 1
Protein (P) and carbohydrate (CHO) ratio of select biomasses.

| | Protein | CHO | Fat | Minerals | Moisture | P:CHO |
|---|---|---|---|---|---|---|
| Vegetable | in fresh | | | | | ratio |
| Garlic | 6.36% | 33.06% | 0.50% | 0.78% | 59.30% | 1:5.2 |
| Onion | 1.10% | 9.34% | 0.10% | 0.21% | 89.25% | 1:8.5 |
| Mushroom | 2.50% | 4.30% | 0.10% | 0.60% | 92.50% | 1:1.7 |
| Spinach | 2.90% | 3.60% | 0.40% | 0.87% | 92.23% | 1:1.2 |
| Kale | 4.30% | 8.80% | 0.90% | 0.82% | 85.18% | 1:2.1 |
| Snow peas | 2.80% | 7.55% | 0.20% | 0.33% | 89.12% | 1:2.7 |
| Asparagus | 2.20% | 3.88% | 0.12% | 0.30% | 93.50% | 1:1.8 |
| Tomatoes | 0.90% | 3.90% | 0.20% | 0.27% | 94.79% | 1:4.3 |
| Avocado | 2.00% | 8.53% | 14.66% | 0.59% | 74.22% | 1:4.3 |
| Carrots | 0.93% | 9.60% | 0.24% | 0.47% | 88.76% | 1:10.3 |
| Broccoli | 2.82% | 6.64% | 0.37% | 0.48% | 89.69% | 1:2.4 |
| Artichoke | 2.89% | 11.39% | 0.34% | 0.72% | 84.66% | 1:3.9 |
| Cauliflower | 1.90% | 5.00% | 0.30% | 0.33% | 92.47% | 1:2.6 |
| Brussel | 3.40% | 9.00% | 0.30% | 0.41% | 86.89% | 1:2.7 |
| sprouts | | | | | | |

In an embodiment, carbohydrate is added to the biomass and/or further biomass to form a protein to carbohydrate ratio of between about 1:1 and 1:10.5. In an embodiment, carbohydrate is added to the biomass and/or further biomass to form a protein to carbohydrate ratio of between about 1:4.5 and 4:1. In an embodiment, carbohydrate is added to the biomass and/or further biomass to form a protein to carbohydrate ratio of between about 1:2.5 and 2:1.

In an embodiment, the biomass may is the entire organism or one or more parts thereof.

In an embodiment, the biomass and/or further biomass comprises the whole biomass (or a piece thereof) in fresh/raw or dried form. In an embodiment, the biomass and/or further biomass is fresh/raw. In an embodiment, the biomass and/or further biomass is pre-treated as described herein.

In an embodiment, the biomass and/or further biomass is a product of an extraction or separation process as described herein suitable for removing one or more component/s from the biomass and/or further biomass.

In an embodiment, the biomass and/or further biomass comprises a bioactive. In an embodiment, the biomass and/or further biomass comprises a bioactive precursor.

In an embodiment, the bioactive and/or bioactive precursor is added to the biomass or further biomass.

In an embodiment, the biomass and/or further biomass is eukaryotic. In an embodiment, the biomass and/or further biomass is prokaryotic (e.g. algae). In an embodiment, the biomass and/or further biomass is from the Plantae or Fungi Kingdom. The material may be any part of a Plantae or Fungi, including where relevant, but not limited to, one or more of leaves, stems, flowers, florets, seeds and roots. In an embodiment, the Plantae is a Brassicaceae. As used herein, “Brassicaceae” refers to members of the Family Brassicaceae commonly referred to as mustards, crucifers or the cabbage family.

In an embodiment, the Brassicaceae is selected from the genus Brassica or Cardamine. In an embodiment, the Brassica is selected from one or more of: Brassica balearica, Brassica carinata, Brassica elongate, Brassica fruticulosa, Brassica hilarionis, Brassica juncea, Brassica napus (rapeseed or canola), Brassica narinosa, Brassica nigra, Brassica oleracea, Brassica perviridis, Brassica rapa, Brassica rupestris, Brassica septiceps, and Brassica tournefortii.

In an embodiment, the Brassica is Brassica oleracea.

In an embodiment, the Brassica is Brassica napus (rapeseed or canola).

In an embodiment, the Brassica selected from one or more of: Brassica oleracea variety oleracea (wild cabbage), Brassica oleracea variety capitate (cabbage), Brassica rapa subsp. chinensis (bok choy), Brassica rapa subsp. pekinensis (napa cabbage), Brassica napobrassica (rutabaga), Brassica rapa var. rapa (turnip), Brassica oleracea variety alboglabra (kai-lan), Brassica oleracea variety viridis (collard greens), Brassica oleracea variety longata (jersey cabbage), Brassica oleracea variety acephala (ornamental kale), Brassica oleracea variety sabellica (kale), Brassica oleracea variety palmifolia (lacinato kale), Brassica oleracea variety ramose (perpetual kale), Brassica oleracea variety medullosa (marrow cabbage), Brassica oleracea variety costata (tronchuda kale), Brassica oleracea variety gemmifera (brussels sprout), Brassica oleracea variety gongylodes (kohlrabi), Brassica oleracea variety italica (broccoli), Brassica oleracea variety botrytis (cauliflower, Romanesco broccoli, broccoli di torbole), Brassica oleracea variety botrytis x italica (broccoflower), and Brassica oleracea variety italicax alboglabra (Broccolini). In an embodiment, the Brassica oleracea is kale.

In an embodiment, the Brassica is Brassica oleracea, variety italica (broccoli).

In an embodiment, the Brassica is Brassica oleracea variety botrytis (cauliflower).

In an embodiment, the Brassica is Brassica oleracea variety gemmifera (brussels sprout).

In an embodiment, the Brassicaceae is selected from one or more of: Cardamine hirsuta (bittercress), Iberis sempervirens (candytuft), Sinapis arvensis (charlock), Armoracia rusticana (horseradish), Pringlea antiscorbutica (kerguelen cabbage), Thlaspi arvense (pennycress), Raphanus raphanistrum subsp. sativus (radish), Eruca sativa (rocket), Anastatica hierochuntica (rose of jericho), Crambe maritima (sea kale), Cakile maritima (sea rocket), Capsella bursa-pastoris (shepherd's purse), sweet alyssum, Arabidopsis thaliana (thale cress), Nasturtium officinale (watercress), Sinapis alba (white mustard), Erophila verna (whitlow grass), Raphanus raphanistrum (wild radish), Isatis tinctoria (woad), and Nasturtium microphyllum (yellow cress).

In an embodiment, the Plantae is Cannabis. In an embodiment, the Cannabis is Cannabis sativa (hemp).

In an embodiment, the Plantae is a fruit or vegetable. In an embodiment, the fruit is selected from one or more of: a simple, aggregate and multiple fruit. In an embodiment, the fruit or vegetable is from the family Umbelliferae, Asparagaceae, Arecaceae, Myrtaceae, Rosaceae, Musaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae and Caesalpinioideae.

In an embodiment, the Umbelliferae is carrot.

In an embodiment, the Asparagaceae is asparagus.

In an embodiment, the Polygonaceae is selected from one or more of: buckwheat, garden sorrel and rhubarb.

In an embodiment, the Cucurbitaceae is selected from one or more of: cucumber, pumpkin, squash and zucchini.

In an embodiment, the fruit is selected from one or more of: apple, apricot, avocado, banana, bilberry, blackberry, blackcurrant, blueberry, coconut, currant, cherry, cherimoya, clementine, cloudberry, damson, durian, elderberry, fig, feijoa, gooseberry, grape, grapefruit, guava, huckleberry, jackfruit, jambul, jujube, kiwifruit, kumquat, lemon, lime, loquat, lychee, mandarin, mango, melon, cantaloupe, honeydew, watermelon, nectarine, orange, passionfruit, paw paw, peach, pear, plum, plumcot, pineapple, pomegranate, pomelo, purple mangosteen, raspberry, rambutan, redcurrant, satsuma, star fruit, strawberry, tangerine, tomato, and ugli fruit.

In an embodiment, the Plantae is a Compositae. In an embodiment, the Compositae is selected from one or more of: artichoke, chamomile, chicory, dandelion, endive, jerusalem artichoke, lettuce, romaine, safflower salsify and sunflower.

In an embodiment, the Plantae is an Amaranthaceae/Chenopodiacae. In an embodiment, the Amaranthaceae/Chenopodiacae is selected from one or more of: amaranth, beet, chard, lamb's-quarters, quinoa, spinach and sugar beet.

In an embodiment, the Plantae is Malvaceae. In an embodiment, the Malvaceae is selected from one or more of: cacao, cotton and okra.

In an embodiment, the Plantae is from the family Amarylidaceae. In an embodiment, the Amarylidaceae is from the subfamily Allioideae. In an embodiment, Allioideae is from the genus Allium. In an embodiment, the Allium is selected from one or more of: Allium sativum (garlic), Allium cepa (onion), Allium ampeloprasum (leeks), Allium schoenoprasum (chives), and Allium oschaninii (shallot).

In an embodiment, the Allium is Allium sativum (garlic).

In an embodiment, the Plantae is from the family Fabaceae. In an embodiment, the Fabaceae is soybean alfalfa, beans, carob, chickpea, green beans, jicama, lentil, pea, snow pea and peanut.

In an embodiment, the Fabaceae is snow pea.

In an embodiment, the Plantae is a cereal. In an embodiment, the cereal is an ancient grain. In an embodiment, the cereal is selected from one or more of: rice, corn, wheat, triticale, barley, millet, sorghum, spelt, oats, freekeh, bulgur, sorghum, farro, einkorn, teff, emmer and/or buckwheat.

In an embodiment, the Plantae is from the Arecaceae family. In an embodiment, the Arecaceae is the coconut palm. In an embodiment, the biomass and/or further biomass is the coconut drupe.

In an embodiment, the Plantae is a grass. In an embodiment, the grass is from the family Poaceae. In an embodiment, the grass is selected from one or more of: bamboo, lemongrass, sugarcane, corn and wheatgrass.

In an embodiment, the Plantae is from the family Camellia sinensis. In an embodiment, the Camellia sinensis is green tea leaves (matcha).

In an embodiment, the Fungi is a mushroom. In an embodiment, the Fungi is from the family Boletaceae, Cantharellaceae, Tricholomataceae, Cortinariaceae, Cantharellaceae, Meripilaceae, Discinaceae, Pleurotaceae, Tricholomataceae and Tuberaceae.

In an embodiment, the Fungi is selected from one or more of: Boletus edulis, Cantharellus cibarius, Cantharellus tubaeformis, Clitocybe nuda, Cortinarius caperatus, Craterellus cornucopioides, Grifola frondosa, Hericium erinaceus, Hydnum repandum, Lactarius deliciosus, Morchella conica var. deliciosa, Morchella esculenta var. rotunda, Pleurotus ostreatus, Tricholoma matsutake, Tuber brumale, Tuber indicum, Tuber macrosporum, Tuber mesentericum, and Tuber aestivum.

In an embodiment, the biomass and/or further biomass is not animal biomass or an animal produced product. In an embodiment, the biomass and/or further biomass is not avian. In an embodiment, the biomass and/or further biomass is not bone or bone marrow. In an embodiment, the biomass and/or further biomass is not animal milk.

In an embodiment, the biomass and/or further biomass is not milk, skim milk or purified milk protein and carbohydrate.

In an embodiment, the biomass and/or further biomass is Plantae or Fungi material that does not meet cosmetic retail standards or is no longer suitable for fresh sale but still edible.

### Bioactives

As used herein “bioactive” refers to a substance having a biological effect. In an embodiment, the bioactive is sensitive to degradation by one or more of oxygen (oxidation), temperature, pH, moisture and light. In an embodiment, the bioactive is an oil, or oil soluble substance.

In an embodiment, the bioactive is selected from one or more of: fatty acid, an isothiocyanate, quercetin, allicin, ajoene, vitamin A, vitamin D, vitamin E, tocopherols, tocotrienols, vitamin K, beta-carotene, lycopene, lutein, zeaxanthin, stigmasterol, beta-sitosterol, campesterol, antioxidants, coenzyme Q10, astaxanthin, cannabinoid, cannabiodiol and a polyphenol.

In an embodiment, the bioactive is selected from one or more of: quercetin, allicin and phenolic acid. In an embodiment, the bioactive is allicin. In an embodiment, the bioactive is ajoene.

In an embodiment, the bioactive is a polyphenol. In an embodiment, the polyphenol is selected from one or more of: catechins, flavonols, flavanols, anthocyanadins, resveratrol, and/or curcumin. Further polyphenols are described herein.

In an embodiment, the bioactive is an isothiocyanate. As used herein “isothiocyanate” refers to sulphur containing phytochemicals with the general structure R—N═C═S which are a product of myrosinase activity upon a glucosinolate and bioactive derivatives therefrom. In an embodiment, the isothiocyanate is sulforaphane (1-isothiocyanato-4-methylsulfinylbutane). In an embodiment, the isothiocyanate is allyl isothiocyanate (3-isothiocyanato-1-propene). In an embodiment, the isothiocyanate is benzyl isothiocyanate. In an embodiment, the isothiocyanate is phenethyl isothiocyanate. In an embodiment, the isothiocyanate is 3-butenyl isothiocyanate. In an embodiment, the isothiocyanate is 5-vinyl-1,3-oxazolidine-2-thione. In an embodiment, the isothiocyanate is 3-(methylthio) propyl isothiocyanate. In an embodiment, the isothiocyanate is 3-(methylsulfinyl)-propyl isothiocyanate. In an embodiment, the isothiocyanate is 4-(methylthio)-butyl isothiocyanate. In an embodiment, the isothiocyanate is 1-methoxyindol-3-carbinol isothiocyanate. In an embodiment, the isothiocyanate is 2-phenylethyl isothiocyanate (also known as phenylethyl isothiocyanate or PEITC). In an embodiment, the isothiocyanate is iberin.

In an embodiment, when the bioactive is an isothiocyanate the biomass and/or further biomass further comprises one or more isothiocyanate bioactive derivative/s or oligomers therefrom. In an embodiment, the isothiocyanate bioactive derivative is a derivative of any of the isothiocyanates as described herein. In an embodiment, the isothiocyanate bioactive derivative is a derivative of sulforaphane. In an embodiment, the isothiocyanate bioactive derivative is indole-3-caribinol. In an embodiment, the isothiocyanate bioactive derivative is methoxy-indole-3-carbinol. In an embodiment, the isothiocyanate bioactive derivative is ascorbigen. In an embodiment, the isothiocyanate bioactive derivative is neoascorbigen.

In an embodiment, the bioactive is a component of the biomass. In an embodiment, the bioactive is a component of the further biomass. In an embodiment, the bioactive is not present in the biomass or further biomass and is added before, during or after preparation of aqueous mixture as described herein. In an embodiment, the bioactive is added before, during or after step ii) of the method as described herein. In an embodiment, the bioactive is added before or during step iii) of the method as described herein. In an embodiment, the bioactive is the oil is step ii) or a component thereof. In an embodiment, the bioactive is a component added to the oil before the oil is added to the aqueous mixture suspension in step ii). In an embodiment, the bioactive is a component infused in the oil before or during step ii). In an embodiment, the bioactive is formed in or after step i) or in step ii).

In an embodiment, the bioactive is a synthetically produced bioactive. In an embodiment, the bioactive is a synthetically produced isothiocyanate. In an embodiment, the bioactive is a synthetically produced sulforaphane.

In an embodiment, when the biomass and/or further biomass comprises: i) Brassicaceae the bioactive is an isothiocyanate; ii) Brassicaceae and the bioactive precursor is a glucosinolate; iii) onion the bioactive is one or more of quercetin, allicin and phenolic acid; iv) garlic the bioactive is one or more of allicin and ajoene; or v) fruit and/or vegetables containing polyphenols. In an embodiment, the Brassicaceae is broccoli the isothiocyanate is sulforaphane. In an embodiment, the bioactive from the biomass and/or further biomass is infused in the oil in step ii) or step iii) of the method as described herein.

In an embodiment, the bioactive is a phytonutrient. As used herein, “phytonutrient” refers to a plant derived substance associated with positive health effects. In an embodiment, the biomass and/or further biomass as described herein comprises one or more phytonutrient/s. In an embodiment, the phytonutrient is selected from one or more of: betalain, indole, organosulfide, phenol, terpene, triterpene, carotenoid, curcuminoid, flavonoids, glucosinolate, isothiocyanate, hydroxycinnamic acid, lignan, lipid, stilbene, sulphide, tocopherol, lutein, zeanthin, isoflavone, flavonoid, coumestna, lycopene, ellagic acid, caffeoylquinic acid, hydroxybenzoic acid, hesperetin, flavonol, terpenoid, phthalide, flavonol, allicin quercetin, sulphide, anthocyanin, resveratrol, and anthoxanthin.

In an embodiment, the phytonutrient is a pigmented phytonutrient. In an embodiment, the pigmented phytonutrient is selected from one or more of: anthocyanin, lutein, zeaxanthin, lycopene, carotenoids and/or anthoxanthin.

In an embodiment, the bioactive is a fatty acid. As used herein, the term “fatty acid” refers to a carboxylic acid (or organic acid), often with a long aliphatic tail, either saturated or unsaturated. Typically fatty acids have a carbon-carbon bonded chain of at least 4 carbon atoms (C4) or at least 8 carbon atoms (C8) in length, more preferably at least 12 carbons in length. Preferred fatty acids of the invention have carbon chains of 18-22 carbon atoms (C18, C20, C22 fatty acids), more preferably 20-22 carbon atoms (C20, C22) and most preferably 22 carbon atoms (C22). Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms. In an embodiment, the fatty acid is esterified to a methyl or ethyl group, such as, for example, a methyl or ethyl ester of a C20 or C22 polyunsaturated fatty acid. Preferred fatty acids are the methyl or ethyl esters of eicosatrienoic acid, docosapentaenoic acid or docosahexaenoic acid, or the mixtures eicosapentaenoic acid and docosahexaenoic acid, or eicosapentaenoic acid, docosapentaenoic acid and docosahexaenoic acid, or eicosapentaenoic acid and docosapentaenoic acid.

In an embodiment, the fatty acid is a polyunsaturated fatty acid. As used herein “polyunsaturated fatty acid” refers to a fatty acid that contains more than one double bond in its backbone. In an embodiment, the polyunsaturated fatty acid is selected from one or more of: an omega-3, omega-6, or omega-9. In an embodiment, the omega-3 is selected from one or more of: hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In an embodiment, the bioactive(s) is one or more or all of eicosapentaenoic acid, docosapentaenoic acid and docosahexaenoic acid. In an embodiment, the omega-6 is selected from one or more of: linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, and tetracosapentaenoic acid. In an embodiment, the omega-9 oil is selected from one or more of: oleic acid, eicosenoic acid, mead acid, erucic acid, and nervonic acid. In an embodiment, the bioactive is a triglyceride.

In an embodiment, the bioactive is an oil.

### Bioactive Precursors

In an embodiment, the biomass and/or further biomass as described herein comprises a bioactive precursor. In an embodiment, a bioactive precursor isadded to the oil as described herein.

In an embodiment, the bioactive precursor is a glucosinolate. As used herein “glucosinolate” refers to a secondary metabolite found at least in the Brassicaceae family that share a chemical structure consisting of a β-D-glucopyranose residue linked via a sulfur atom to a (Z)-N-hydroximinosulfate ester, plus a variable R group derived from an amino acid as described in Halkier and Gershenzon (2006). Examples of glucosinolates are provided in Halkier and Gershenzon (2006) and Agerbirk and Olsen (2012). The hydrolysis of glucosinolate can produce isothiocyanates, nitriles, epithionitrile, thiocyanate and oxazolidine-2-thione. Many glucosinolates play a role in plant defence mechanisms against pests and disease.

Glucosinolates are stored in Brassicaceae in storage sites. As used herein, a “storage site” is a site within the Brassicaceae where glucosinolates are present and myrosinase is not present.

As used herein “myrosinase” also referred to as “thioglucosidase”, “sinigrase”, or “sinigrinase” refers to a family of enzymes (EC 3.2.1.147) involved in plant defence mechanisms that can cleave thio-linked glucose. Myrosinases catalyze the hydrolysis of glucosinolates resulting in the production of isothiocyanates. Myrosinase is stored sometimes as myrosin grains in the vacuoles of particular idioblasts called myrosin cells, but have also been reported in protein bodies or vacuoles, and as cytosolic enzymes that tend to bind to membranes.

In an embodiment, pre-treating as described herein improves the access of myrosinase to a glucosinolate producing the isothiocyanate bioactive. As used herein “improves the access” or “access is improved” refers to increasing the availability of glucosinolate to the myrosinase enzyme allowing for the production of an isothiocyanate. In an embodiment, access is improved by the release of a glucosinolate from a glucosinolate storage site. In an embodiment, the glucosinolate storage site is mechanically ruptured (i.e. by maceration). In an embodiment, access is improved by allowing the entry of myrosinase into a glucosinolate storage site. In an embodiment, access is improved by the release of myrosinase from myrosin cells. In an embodiment, about 10% to about 90% of a glucosinolate is released from a glucosinolate storage site. In an embodiment, about 20% to about 80% of a glucosinolate is released from a glucosinolate storage site. In an embodiment, about 30% to about 70% of a glucosinolate is released from a glucosinolate storage site. In an embodiment, about 40% to about 60% of a glucosinolate is released from a glucosinolate storage site. In an embodiment, about 45% to about 55% of a glucosinolate is released from a glucosinolate storage site. In an embodiment, about 10% of a glucosinolate is released from a glucosinolate storage site. In an embodiment, about 20% of a glucosinolate is released from a glucosinolate storage site. In an embodiment, about 30% of a glucosinolate is released from a glucosinolate storage site. In an embodiment, about 40% of a glucosinolate is released from a glucosinolate storage site. In an embodiment, about 50% of a glucosinolate is released from a glucosinolate storage site. In an embodiment, about 60% of a glucosinolate is released from a glucosinolate storage site. In an embodiment, about 70% of a glucosinolate is released from a glucosinolate storage site. In an embodiment, about 80% of a glucosinolate is released from a glucosinolate storage site. In an embodiment, about 90% of a glucosinolate is released from a glucosinolate storage site.

In an embodiment, the glucosinolate/s is selected from one or more of: an aliphatic, indole or aromatic glucosinolate.

In an embodiment, the aliphatic glucosinolate is selected from one or more of: glucoraphanin (4-Methylsulphinylbutyl or glucorafanin), sinigrin (2-Propenyl), gluconapin (3-Butenyl), glucobrassicanapin (4-Pentenyl), progoitrin (2 (R)-2-Hydroxy-3-butenyl, epiprogoitrin (2 (S)-2-Hydroxy-3-butenyl), gluconapoleiferin (2-Hydroxy-4-pentenyl), glucoibervirin (3-Methylthiopropyl, glucoerucin (4-Methylthiobutyl), dehydroerucin (4-Methylthio-3-butenyl, glucoiberin (3-Methylsulphinylpropyl), glucoraphenin (4-Methylsulphinyl-3-butenyl), glucoalyssin (5-Methylsulphinylpentenyl), and glucoerysolin (3-Methylsulphonylbutyl, 4-Mercaptobutyl).

In an embodiment, the indole glucosinolate is selected from one or more of: glucobrassicin (3-Indolylmethyl), 4-hydroxyglucobrassicin (4-Hydroxy-3-indolylmethyl), 4-methoxyglucobrassicin (4-Methoxy-3-indolylmethyl), and neoglucobrassicin (1-Methoxy-3-indolylmethyl).

In an embodiment, the indole glucosinolate is selected from one or more of: Glucotropaeolin (Benzyl) and Gluconasturtiin (2-Phenylethyl).

In an embodiment, the glucosinolate is selected from one or more of: benzylglucosinolate, allylglucosinolate and 4-methylsulfinylbutyl. In an embodiment, the glucosinolate is glucoraphanin (4-Methylsulphinylbutyl). In an embodiment, the glucosinolate is glucobrassicin (3-Indolylmethyl).

In an embodiment, the glucosinolate is converted to an isothiocyanate before or during step i), ii) and or iii) of the method of producing a powder as described herein.

In an embodiment, the bioactive precursor is a component of the biomass. In an embodiment, the bioactive precursor is a component of the further biomass. In an embodiment, the bioactive precursor is not present in the biomass or further biomass and is added before, during or after preparation of aqueous mixture as described herein. In an embodiment, the bioactive precursor is added before, during or after step ii) of the method as described herein. In an embodiment, the bioactive precursor is added before or during step iii) of the method as describes herein. In an embodiment, the bioactive precursor is the oil or a component thereof in step ii). In an embodiment, the bioactive precursor is a component added to the oil before the oil is added to the aqueous mixture in step ii). In an embodiment, the bioactive precursor is a component infused in the oil before or during step ii).

### Pre-Treatment

In an embodiment, the biomass and/or further biomass as described herein is pre-treated. As used herein “pre-treatment” or “pre-treating” or “pre-treated” refers to processing of the biomass and/or further biomass to break the material into smaller components, remove a component (e.g. remove a specific component not suitable for ingestion or extract a specific component for a different use e.g. oil) or modify a component of the biomass and/or further biomass. In an embodiment, modifying a component includes, for example, producing a bioactive, or producing an oligosaccharide or a polysaccharide. In an embodiment, pre-treating does not alter the ratio of protein to carbohydrate in the biomass or further biomass.

In an embodiment, pre-treating comprises one or more of the following: i) heating; ii) macerating; iii) microwaving; iv) exposure to low frequency sound waves (ultrasound); v) pulse electric field processing; vi) static high pressure; vii) extrusion; viii); enzyme treatment; ix) fermentation; x) an extraction or separation process; and xi) drying.

In an embodiment, the biomass and/or further biomass is heated in a fuel based heating system, an electricity based heating system (i.e. an oven or ohmic heating), radio frequency heating, high pressure thermal processing, ultra high temperature (UHT) treatment plant, in a retort or a steam based heating system (indirect or direct application of steam). In an embodiment, the biomass and/or further biomass is heated in an oven, water bath, bioreactor, stove, water blancher, or steam blancher. In an embodiment, the biomass and/or further biomass is heated via high pressure thermal heating. In an embodiment, the biomass and/or further biomass is heated via ohmic heating. In an embodiment, the biomass and/or further biomass is heated via radio frequency heating. In an embodiment, the biomass and/or further biomass is heated via high pressure thermal processing. In an embodiment, the biomass and/or further biomass is placed in a sealed pack or container for high pressure thermal processing.

In an embodiment, pre-treating comprises heating the biomass and/or further biomass to about 50° C. to about 140° C. In an embodiment, heating comprises heating the biomass and/or further biomass to about 55° C. to about 70° C. In an embodiment, heating comprises heating the biomass and/or further biomass to about 60° C. to about 70° C. In an embodiment, heating comprises heating the biomass and/or further biomass to about 65° C. to about 70° C. In an embodiment, heating comprises heating the biomass and/or further biomass to about 70° C. to about 140° C. In an embodiment, heating comprises heating the biomass and/or further biomass to about 80° C. to about 130° C. In an embodiment, heating comprises heating the biomass and/or further biomass to about 90° C. to about 120° C. In an embodiment, heating comprises heating the biomass and/or further biomass to about 100° C. to about 110° C. In an embodiment, heating comprises heating the biomass and/or further biomass to about 75° C. for about 2 minutes. In an embodiment, heating comprises heating the biomass and/or further biomass to about 100° C. for about 30 minutes. In an embodiment, pre-treatment comprises heating at the lower end of the above temperature range for a longer period of time or treatment at the higher end of the above temperature range for a short period of time.

In an embodiment, heating comprises steaming the biomass and/or further biomass. In an embodiment, the biomass and/or further biomass is steamed to a temperature of about 100° C. In an embodiment, the biomass and/or further biomass is steamed for at least about 30 seconds. In an embodiment, the biomass and/or further biomass is steamed for at least 1 minute. In an embodiment, the biomass and/or further biomass is steamed for at least 2 minutes. In an embodiment, the biomass and/or further biomass is steamed for at least 3 minutes. In an embodiment, the biomass and/or further biomass is steamed for at least 4 minutes. In an embodiment, the biomass and/or further biomass is steamed for at least 5 minutes. In an embodiment, the biomass and/or further biomass is steamed to a temperature of about 100° C. for 30 minutes.

In an embodiment, heating comprises ultra high temperature (UHT) treatment of the biomass and/or further biomass. In an embodiment, the biomass and/or further biomass is UHT treated at a temperature of about 140° C.

In an embodiment, heating comprises retorting of the biomass and/or further biomass. In an embodiment, the biomass and/or further biomass is retorted at a temperature of about 116° C. to about 130° C.

In an embodiment, pre-treating comprises macerating the biomass and/or further biomass. In an embodiment, the biomass and/or further biomass is macerated with a shredder, blender, colloid mill, grinder or pulveriser. In an embodiment, the biomass and/or further biomass is macerated so that at least 80% of the biomass and/or further biomass is of a size of 2 mm or less. In an embodiment, the biomass and/or further biomass is macerated so that at least 80% of the biomass and/or further biomass is of a size of 1 mm or less. In an embodiment, the biomass and/or further biomass is macerated so that at least 80% of the biomass and/or further biomass is of a size of 0.5 mm or less. In an embodiment, the biomass and/or further biomass is macerated so that at least 80% of the biomass and/or further biomass is of a size of 0.25 mm or less. In an embodiment, the biomass and/or further biomass is macerated so that at least 80% of the biomass and/or further biomass is of a size of 0.1 mm or less. In an embodiment, the biomass and/or further biomass is macerated so that at least 80% of the biomass and/or further biomass is of a size of 0.05 mm or less. In an embodiment, the biomass and/or further biomass is macerated so that at least 80% of the biomass and/or further biomass is of a size of 0.025 mm or less. In an embodiment, the biomass and/or further biomass is heated during maceration. In some embodiments, heating facilitates the conversion of bioactive precursors into bioactive, such as for example, sulforaphane and ajoene. In an embodiment, the biomass and/or further biomass is heated to a temperature of about 25° C. to about 80° C. during maceration. In an embodiment, the biomass and/or further biomass is heated to a temperature of about 40° C. to about 70° C. during maceration. In an embodiment, the biomass and/or further biomass is heated to a temperature of about 50° C. to about 70° C. during maceration. In an embodiment, the biomass and/or further biomass is heated to a temperature of about 60° C. to about 70° C. during maceration. In an embodiment, the biomass and/or further biomass is heated to a temperature of about 70° C. during maceration for about 2 to about 5 mins. In an embodiment, the biomass and/or further biomass is heated to a temperature of about 30° C. to about 80° C. during maceration for about 1 to about 5 hours.

In an embodiment, pre-treating comprises heating and macerating the biomass and/or further biomass.

A person skilled in the art will appreciate that “microwaves” or “microwaving” heats a substance such as biomass and/or further biomass by passing microwave radiation through the substance. In an embodiment, pre-treating comprises microwaving the biomass and/or further biomass. In an embodiment, biomass and/or further biomass is pre-treated in a consumer microwave or industrial microwave. In an embodiment, the industrial microwave is a continuous microwave system, for example, but not limited to the MIP 11 Industrial Microwave Continuous Cooking Over (Ferrite Microwave Technologies). In an embodiment, pre-treating comprises microwaving the biomass and/or further biomass. In an embodiment, the biomass and/or further biomass is microwaved at about 0.9 to about 2.45 GHz. In an embodiment, the biomass and/or further biomass is microwaved for at least 30 seconds, or at least 1 minute, or at least 2 minutes, or at least 3 minutes. In an embodiment, microwaving increases the temperature of the biomass and/or further biomass to about 70 to about 80° C., preferably about 76° C.

In an embodiment, pre-treating comprises exposing the biomass and/or further biomass at low to medium frequency ultrasound waves. In an embodiment, pre-treating comprises exposing the biomass and/or further biomass to thermosonication (low to medium frequency ultrasound waves with heat of about 50° C. to about 140° C.). In an embodiment, the ultrasound waves are generated with an industrial scale ultrasonic processor. In an embodiment, the ultrasonic processor is a continuous or batch ultrasonic processor. In an embodiment, the ultrasonic processor is for example, but not limited to, UIP500 hd or UIP4000 (Hielscher, Ultrasound Technology). In an embodiment, the ultrasounds waves are at a frequency of about 20 kHz to about 600 kHz. In an embodiment, the biomass and/or further biomass is exposed to sound waves for at least 30 seconds, or at least 1 minute, or at least 2 minutes, or at least 3 minutes, or about 5 minutes, or about 6 minutes, or about 7 minutes, or about 7.5 minutes, or about 8 minutes.

In an embodiment, pre-treating comprises exposing the biomass and/or further biomass to pulse electric field processing. Pulse electric field processing is a non-thermal processing technique comprising the application of short, high voltage pulses. The pulses induce electroporation of the cells of the biomass and/or further biomass. In an embodiment, pulse electric field processing heats the biomass and/or further biomass to a temperature of about 50 to about 140° C. In an embodiment, pulse electric field processing heats the biomass and/or further biomass to a temperature of about 70° C. to about 110° C. In an embodiment, pulse electric field processing heats the biomass and/or further biomass to a temperature of about 80° C. to about 100° C. In an embodiment, pulse electric field processing comprises treating the biomass and/or further biomass with voltage pulses of about 20 to about 80 kV.

In an embodiment, pre-treating comprises hydrostatic pressure. In an embodiment, hydrostatic pressure comprises treating the biomass and/or further biomass with about 100 to about 600 MPa.

In an embodiment, pre-treating comprises extrusion. In an embodiment, extrusion comprises applying a force to the biomass or product, usually at elevated temperature and/or high pressure through a barrel prior to expulsion of the mass through an orifice. In an embodiment, the high temperatures, high pressures and mechanical forces applied during extrusion modify the functional properties of the material. In an embodiment, the extrusion process is carried out using a co-rotating twin screw extruder (MPF 18:25, APV Baker Ltd., Peterborough, UK) or a lab-scale, co-rotating and intermeshed twin-screw lab extruder (KDT30-II, Jinan Kredit Machinery Co. Ltd., China). In an embodiment, the extrusion process produces Maillard reaction products.

In an embodiment, pre-treating comprises enzyme treatment to transform one or more components in the biomass and/or further biomass to a new component. For example, the enzyme converts simple sugars into oligosaccharides or polysaccharides. In an embodiment, the enzyme is selected from one or more of a: glycosyltransferase, ii) glycosidase, iii) pectinase, iv) esterase, v), oxidoreductase, vi) protease, vii) pectinase, viii) polygalacturonase, ix) amylase and x) pullulanase. In an embodiment, the glycosyltransferase is selected from one or more or all of a: i) dextransucrase, ii) alternansucrase, and iii) fructosyltransferases. In an embodiment, the fructosyltransferases is for example levansucrase, and/or inulosucrase. In an embodiment, the oxidoreductase is mannitol dehydrogenase.

In an embodiment, pre-treating comprises fermenting the biomass and/or further biomass. As used herein “fermentation” refers to the biochemical breakdown of the biomass and/or further biomass by bacteria, such as for example lactic acid and/or acetic acid bacteria. As used herein “lactic bacteria” or “lactic acid bacteria” are bacteria that produce lactic acid as the main product of carbohydrate fermentation. As used herein “acetic bacteria” or “acetic acid bacteria” are bacteria that produce acetic acid as an end product of carbohydrate fermentation.

In an embodiment, lactic acid and/or acetic acid bacteria produce enzymes that catalyze the production of mannitol, oligosaccharides and/or polysaccharides. In an embodiment, the lactic acid bacteria is from one or more of the Genera Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and/or Weissella. In an embodiment, the lactic acid bacteria is selected from one or more of: Leuconostoc mesenteroides, Lactobacillus reuteri and/or Lactobacillus gasseri.

Leuconostoc mesenteroides are gram positive, epiphytic bacteria (McCleskey et al., 1947). Leuconostoc mesenteroides also produce the antimicrobial proteins bacteriocins, which are used in the meat industry as natural preservatives. In an embodiment, the lactic acid bacteria is Leuconostoc mesenteroides. In an embodiment, the Leuconostoc mesenteroides is selected from ATCC 8293 (equivalent to NRRL B-1118) and/or NRRL B-512F investigated in Olvera et al. (2007).

In an embodiment, the acetic acid bacteria is from Acetobacteraceae. In an embodiment, the Acetobacteraceae is Gluconacetobacter.

In an embodiment, the biomass and/or further biomass comprises fermentation for about 8 hours to about 30 hours. In an embodiment, fermentation is for at least 8 hours. In an embodiment, fermentation is for at least 10 hours. In an embodiment, fermentation is for at least 15 hours. In an embodiment, fermentation is for at least 20 hours. In an embodiment, fermentation is for at least 24 hours. In an embodiment, fermentation is for at least 30 hours.

In an embodiment, fermentation is at a pH of about 5 to about 7. In an embodiment, fermentation is at a pH of about 5.3. In an embodiment, the material from step i) is at a pH of about 4 at the end of fermentation. In an embodiment, fermentation is at a temperature of about 24° C. to about 36° C. In an embodiment, fermentation is at a temperature of about 28° C. to about 32° C. In an embodiment, fermentation is at a temperature of about 30° C.

In an embodiment, pre-treatment releases or aids in the release of a glucosinolate from glucosinolate storage site and/or allows myrosinase to enter a glucosinolate storage site in the biomass and/or further biomass. In an embodiment, pre-treating increases the exposure of a glucosinolate to myrosinase allowing myrosinase to convert a glucosinolate to an isothiocyanate.

In an embodiment, pre-treating converts about 10% to about 90% of a glucosinolate to an isothiocyanate. In an embodiment, pre-treating converts about 20% to about 80% of a glucosinolate to an isothiocyanate. In an embodiment, pre-treating converts about 30% to about 70% of a glucosinolate to an isothiocyanate. In an embodiment, pre-treating converts about 40% to about 60% of a glucosinolate to an isothiocyanate. In an embodiment, pre-treating converts about 10% of a glucosinolate to an isothiocyanate. In an embodiment, pre-treating converts about 20% of a glucosinolate to an isothiocyanate. In an embodiment, pre-treating converts about 30% of a glucosinolate to an isothiocyanate. In an embodiment, pre-treating converts about 40% of a glucosinolate to an isothiocyanate. In an embodiment, pre-treating converts about 50% of a glucosinolate to an isothiocyanate. In an embodiment, pre-treating converts about 60% of a glucosinolate to an isothiocyanate. In an embodiment, pre-treating converts about 70% of a glucosinolate to an isothiocyanate. In an embodiment, pre-treating converts about 80% of a glucosinolate to an isothiocyanate. In an embodiment, pre-treating converts about 90% of a glucosinolate to an isothiocyanate.

In an embodiment, pre-treating comprises treating the biomass and/or further biomass with an extraction or separation process to reduce the amount of one or more components in the biomass and/or further biomass (e.g. the biomass may be canola meal where the canola oil has been removed or partially removed). In an embodiment, the other components are suitable for producing other products or are non-edible or poor tasting components of the biomass and/or further biomass.

In an embodiment, the extraction or separation process is for the removal of a component selected from oil, bioactive or bioactive precursor, polyphenols, carotenoids, or juice from the biomass. In an embodiment, the extraction or separation process produces canola meal, nut meal, soybean meal, coconut meal, palm kernel meal, hemp oil press cakes, chia oil seed cake or rice bran which may be used as a biomass in the methods as described herein. In an embodiment, the extraction or separation process produces pomace (e.g. olive or apple pomace) which may be used as a biomass in the methods as described herein. In an embodiment, the extraction or separation process may comprises removing a non-edible component from the biomass (e.g. seeds or stalks). In an embodiment, the extraction or separation process may comprise grinding, cutting, milling, centrifugation and/or filtration.

As used herein “reduced” means that the level of a component is lower in the biomass or further biomass after treatment with the extraction process than in the biomass or further biomass before treatment with the extraction process.

In an embodiment, the level of the component is reduced from about 5% to about 90%. In an embodiment, the level of the component is reduced by about 5%. In an embodiment, the level of the component is reduced by about 10%. In an embodiment, the level of the component is reduced by about 15%. In an embodiment, the level of the component is reduced by about 20%. In an embodiment, the level of the component is reduced by about 30%. In an embodiment, the level of the component is reduced by about 40%. In an embodiment, the level of the component is reduced by about 50%. In an embodiment, the level of the component is reduced by about 60%. In an embodiment, the level of the component is reduced by about 70%. In an embodiment, the level of the component is reduced by about 80%. In an embodiment, the level of the component is reduced by about 90%. In an embodiment, the level of the component is reduced by about 100%.

In an embodiment, pre-treating comprises drying or partially drying the biomass. In an embodiment, drying comprises tray drying, drum drying, roller drying, fluid bed drying, impingement drying, spray drying, freeze-drying (lyophilisation or cryodesiccation), thin-film belt dryer, vacuum microwave drying, ultrasonic-assisted drying, extrusion porosification technology or any other method known to a person skilled in the art. In an embodiment, pre-treating comprises freeze-drying the biomass. In an embodiment, pre-treating comprises heating then freeze-drying the biomass. In an embodiment, pre-treating comprises drum drying. In an embodiment pre-treating comprises spray drying.

A person skilled in the art would appreciate that pre-treating does not comprise separately purifying protein and purifying carbohydrate from the biomass.

In an embodiment, pre-treating does not alter the ratio of protein:carbohydrate in the biomass.

### Preparation of an Aqueous Mixture

As described herein a method of the invention comprises obtaining an aqueous mixture from a biomass from a first species of organism comprising protein and carbohydrate. As used herein “aqueous mixture” refers to a mixture comprising the biomass with water. In an embodiment, the aqueous mixture further comprises protein and carbohydrate from at least one further biomass from a species of organism (e.g. a second, third, fourth, fifth etc.). In an embodiment, the aqueous mixture is homogenous. In an embodiment, the aqueous mixture is a suspension.

In an embodiment, the method further comprises forming the aqueous mixture. In an embodiment, the aqueous mixture is formed by combining water at a temperature of about 40° C. to about 100° C. with the biomass and optionally the further biomass. In an embodiment, the aqueous mixture is formed by combining water at a temperature of about 40° C. to about 80° C. with the biomass and optionally the further biomass. In an embodiment, the aqueous mixture is formed by combining water at a temperature of about 45° C. to about 70° C. with the biomass and optionally the further biomass. In an embodiment, the aqueous mixture is formed by combining water at a temperature of about 55° C. to about 65° C. with the biomass and optionally the further biomass. In an embodiment, the aqueous mixture is formed by combining water at a temperature of about 60° C. with the biomass and optionally the further biomass.

In an embodiment, the aqueous mixture comprises a bioactive and/or a bioactive precursor. In an embodiment, a bioactive and/or bioactive precursor present in the biomass and/or further biomass is present in the aqueous mixture in a form suitable for infusion into the oil in step ii) or iii) of the method as described herein.

In an embodiment, a bioactive and/or bioactive precursor suitable for infusion into the oil in step ii) or iii) of the method is added to the aqueous mixture.

In an embodiment, a mineral is added to the biomass before preparation of the aqueous mixture or in step i) or ii) of the method as described herein. In an embodiment, the mineral is selected from one or more of: zinc, calcium, magnesium, selenium and chromium.

### Lipid

In an embodiment, a method as described herein further comprises the addition of a lipid. As used herein “lipid” refers to a ester of a long straight-chain carboxylic acid that is insoluble in water but soluble in an organic solvent. In an embodiment, the lipid is saponifiable. In an embodiment, the lipid is an oil as described herein. In an embodiment, the lipid is a wax as described herein.

### Oils

In an embodiment, a method as described herein further comprises the addition of oil to the aqueous mixture. As used herein “oil” refers to a viscous liquid that is hydrophobic and lipophilic and not miscible with water. In an embodiment, the oil is susceptible to deterioration by one or more of oxidation, temperature, pH, moisture and light. In an embodiment, the oil is a bioactive.

In an embodiment, the oil comprises a fatty acid as described herein. In an embodiment, the oil comprises a polyunsaturated fatty acid as described herein. In an embodiment, the polyunsaturated fatty acid is selected from one or more of: an omega-3, omega-6, or omega-9 fatty acid. In an embodiment, the omega-3 is selected from one or more of: hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In an embodiment, the omega-6 is selected from one or more of: linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, and tetracosapentaenoic acid. In an embodiment, the omega-9 is selected from one or more of: oleic acid, eicosenoic acid, mead acid, erucic acid, and nervonic acid.

In an embodiment, the oil is a Plantae oil. In an embodiment, the oil is a vegetable oil. In an embodiment, the oil is an animal oil. In an embodiment, the animal oil is a marine oil or fish oil.

In an embodiment, the oil is selected from one or more of: fish oil, krill oil, marine oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, medium chain triglycerides (MCT) and grapeseed oil. In an embodiment, the canola oil comprises one or more long chain polyunsaturated fatty acids such as eicosapentaenoic acid (EPA), docosapentaenoic acid (DPA) and docosahexaenoic acid (DHA) which can be obtained from transgenic Brassica encoding the required elongases and desaturases (see, for example, WO 2015/089587).

In an embodiment, the fish oil is selected from one or more of: tuna oil, herring oil, mackerel oil, anchovy oil, sardine oil, cod liver oil, and shark oil.

In an embodiment, the essential oil is selected from one or more of: oregano oil, mint oil, basil oil, rosemary oil, tea tree oil, time oil, camphor oil, cardamon oil, citrus oil, clove oil, and/or saffron oil.

In an embodiment, the oil comprises dairy fats.

In an embodiment, the oil is olive oil.

In an embodiment, the oil is sunflower oil.

In an embodiment, the oil is canola oil.

In an embodiment, the oil comprises one or more bioactive/s and/or bioactive precursor/s. Thus, in some embodiments, the oil acts as a bioactive carrier. In an embodiment, the bioactive and/or bioactive precursor is added to the oil before the oil is added to the aqueous mixture. In an embodiment, the bioactive and/or bioactive precursor is infused in oil in step ii) of the method as described herein. In an embodiment, the bioactive and/or a bioactive precursor is infused in oil in step iii) of the method as described herein. In an embodiment, the bioactive and/or bioactive precursor is from the biomass and/or further biomass as described herein. In an embodiment, the bioactive and/or bioactive precursor is not from the biomass and/or further biomass.

### Wax

In an embodiment, the method as described herein further comprises the addition of a wax. As used herein “wax” or “waxes” are esters of long chain saturated and unsaturated fatty acids with long chain alcohols. In an embodiment, the long chain saturated fatty acid is a C14 to C26. In an embodiment, the unsaturated fatty acid with long chain alcohols is a C16 to C30. In an embodiment, the wax is selected from one or more of: candellila wax, carnauba wax, beeswax, rice bran wax, sugar cane wax and sunflower wax.

### Preparation of an Emulsion or Suspension

As used herein “emulsion” refers to a dispersion of droplets/particles of one liquid in another in which it is not soluble or miscible. In one embodiment, the droplets are oil dispersed in the aqueous mixture. In an embodiment, the emulsion is a wet emulsion. In an embodiment, the emulsion is dried into powder. In an embodiment, the emulsion is extruded. In an embodiment, the emulsion is extruded with a powder matrix.

In an embodiment, oil droplets produced by the methods described herein are about 0.2 μm to about 10 μm. In an embodiment, oil droplets produced by the methods described herein are about 1 μm to about 10 μm. In an embodiment, oil droplets produced by the methods described herein are about 2 μm to about 8 μm. In an embodiment, oil droplets produced by the methods described herein are about 2 μm to about 4 μm.

In an embodiment, the mean oil droplet size is about 0.2 μm to about 10 μm. In an embodiment, the mean oil droplet size is about 1 μm to about 10 μm. In an embodiment, the mean oil droplet size is about 2 μm to about 8 μm. In an embodiment, the mean oil droplet size is about 2 μm to about 4 μm.

As used herein “suspension” refers to dispersion of droplets/particles of one substance throughout the bulk of another substance. In one embodiment, the droplets are oil dispersed in the aqueous mixture.

As used herein producing or forming an emulsion or suspension refers to entrapment or encapsulation of a substance in the aqueous mixture reducing the exposure of the substance to degradation. In an embodiment, the substance is oil. In an embodiment, the substance is a bioactive. In an embodiment, the bioactive is a fatty acid.

In an embodiment, the oil is heated when it is added to the aqueous mixture in step ii) as described herein. In an embodiment, the oil is heated to about 30° C. to about 80° C. In an embodiment, the oil is heated to about 40° C. to about 70° C. In an embodiment, the oil is heated to about 45° C. to about 65° C. In an embodiment, the oil is heated to about 50° C. to about 60° C.

In an embodiment, a bioactive and/or bioactive precursor is added to the oil before it is added to the aqueous mixture. In an embodiment, a bioactive and/or bioactive precursor is added to the aqueous mixture before, during or after addition of the oil to the aqueous mixture.

In an embodiment, forming an emulsion or suspension as described in step iii) comprises mixing of the oil and aqueous mixture. In an embodiment, mixing comprises agitation under high shear. In an embodiment, mixing comprises homogenization to obtain a small oil droplet size. In an embodiment, oil droplets produced by homogenization are about 0.2 μm to about 10 μm in diameter. In an embodiment, oil droplets produced by homogenization are about 1 μm to about 10 μm in diameter. In an embodiment, oil droplets produced by homogenization are about 2 μm to about 8 μm in diameter. In an embodiment, oil droplets produced by homogenization are about 2 μm to about 4 μm in diameter. In an embodiment, homogenization forms a homogenous emulsion.

In an embodiment, one or more bioactive/s and/or bioactive precursor/s are present in the aqueous solution infuse into the oil before or during step ii) or step iii) of the method as described herein.

In an embodiment, a bioactive and/or bioactive precursor entrapped or encapsulated in the emulsion or suspension by the methods described herein are less susceptible to oxygen degradation than the same bioactive and/or bioactive precursor entrapped or encapsulated by the MicroMAX® encapsulation method (WO01/74175).

In an embodiment, the oil content of emulsion or suspension is from about 1% to about 10% w/v. In an embodiment, the oil content of emulsion or suspension is from about 1.2% to about 9% w/v. In an embodiment, the oil content of emulsion or suspension is from about 1.3% to about 8% w/v. In an embodiment, the oil content of emulsion or suspension is from about 1.4% to about 7% w/v. In an embodiment, the oil content of emulsion or suspension is from about 1.5% to about 6% w/v.

In an embodiment, about 5% w/w to about 50% of the oil is entrapped or encapsulated in the biomass after the emulsion or suspension is dried. In an embodiment, about 10% w/w to about 50% of the oil is entrapped or encapsulated in the biomass after the emulsion or suspension is dried. In an embodiment, about 20% w/w to about 40% of the oil is entrapped or encapsulated in the biomass after the emulsion or suspension is dried. In an embodiment, the emulsion comprises a dispersed probiotic.

### Post-Treating

In an embodiment, the method as described herein comprises post-treating the emulsion or suspension to reduce microbial activity.

As used herein “post-treatment”, “post-treated” or “post-treating” refers to treatment of the emulsion or suspension as described herein to reduce microbial organisms.

A person skilled in the art will appreciate that the post treatment is any method that inactivates microbes or alters the product characteristics (e.g. stability as well as physical structure), including for example, one or more of heat treatment (including pasteurisation), microwaving, ultrasound, UV treatment, high pressure processing, ultra-high temperature processing (UHT) and retorting.

In an embodiment, the emulsion or suspension is post-treated with heat processing. In an embodiment, the emulsion or suspension is post-treated with high pressure processing. In an embodiment, the emulsion or suspension is in a sealed package during post-treatment. In an embodiment, the emulsion or suspension is in a sealed package during high pressure processing. In an embodiment, the emulsion or suspension is in a sealed package during heat treatment. In an embodiment, high pressure processing comprises treating the emulsion or suspension with isostatic pressure at about 100 to about 600 MPa. In an embodiment, high pressure processing comprises treating the emulsion or suspension with isostatic pressure at about 350 to about 550 MPa. In an embodiment, high pressure processing comprises treating the emulsion or suspension with isostatic pressure at about 300 to about 400 MPa. In an embodiment, high pressure process is for about 1 minute, or about 2 minutes, or about 3 minutes or about 4 minutes at about 25° C. In an embodiment, heat treatment comprises heating the microparticle to a temperature of about 60° C. to about 80° C. In an embodiment, heat treatment comprises heating the emulsion or suspension to a temperature of about 65° C. to about 75° C. In an embodiment, heat treatment comprises heating the emulsion or suspension under retort (120° C.). In an embodiment, heat treatment comprises heating the emulsion or suspension under UHT conditions (>120-140° C.).

In embodiment, post treatment comprises microwaving. In an embodiment, microwaving comprises treatment with about 750 W for about 1 minute, or about 2 minutes, or about 2.5 minutes, or about 3 minutes. In an embodiment, microwaving increases the temperature of the biomass and/or further biomass to about 70 to about 80° C., preferably about 76° C.

### Preparation of Powders

In an embodiment, the emulsion or suspension as described herein is partially dried or dried to reduce the water content. In an embodiment, the method as described herein comprises drying the emulsion or suspension to reduce the water content to about 1 to about 14%. In an embodiment, the method as described herein comprises drying the emulsion or suspension to reduce the water content to about 1 to about 13%. In an embodiment, the method comprises drying the emulsion or suspension to reduce the water content to about 1 to about 12%. In an embodiment, the method comprises drying the emulsion or suspension to reduce the water content to about 1 to about 10%. In an embodiment, the method comprises drying the emulsion or suspension to reduce the water content to about 2 to about 8%. In an embodiment, the method comprises drying the emulsion or suspension to reduce the water content to about 2 to about 6%. In an embodiment, the method comprises drying the emulsion or suspension to reduce the water content to about 2 to about 4%. In an embodiment, the method comprises drying the emulsion or suspension to reduce the water content to about 2 to about 3%.

In an embodiment, the method as described herein comprises drying the emulsion or suspension to reduce the water activity to a low water activity to about 0.1 to about 0.7. In an embodiment, the method comprises drying the emulsion or suspension to reduce the water activity to a low water activity to about 0.2 to about 0.6. In an embodiment, the method comprises drying the emulsion or suspension to reduce the water activity to a low water activity to about 0.2 to about 0.5. In an embodiment, the method comprises drying the emulsion or suspension to reduce the water activity to a low water activity to about 0.3 to about 0.4. In an embodiment, the method comprises drying the emulsion or suspension to reduce the water activity to a low water activity of about 0.4.

In an embodiment, the method as described herein comprises drying the emulsion or suspension to form a powder. Drying may include for example spray drying, freeze-drying (lyophilisation or cryodesiccation), tray drying, drum drying, roller drying, fluid bed drying, impingement drying, refractance windows drying, thin-film belt drying, vacuum microwave drying, ultrasonic-assisted drying, extrusion porosification technology or any other method known to a person skilled in the art.

In an embodiment, the emulsion or suspension is dried to produce a mean dry particle size of about 10 μM to about 4000 μM. In an embodiment, the emulsion or suspension is dried to produce a mean dry particle size of about 10 μM to about 3000 μM. In an embodiment, the emulsion or suspension is dried to produce a mean dry particle size of about 20 μM to about 2000 μM. In an embodiment, the emulsion or suspension is dried to produce a mean dry particle size of about 10 μM to about 1000 μM. In an embodiment, the emulsion or suspension is dried to produce a mean dry particle size of about 10 μM to about 500 μM.

In an embodiment, the emulsion or suspension is dried by spray drying (e.g. a Drytec laboratory spray dryer) to form a powder. For example, the emulsion or suspension is dried using a Drytec laboratory spray dryer with a rotary atomiser, ultrasonic nozzle or twin fluid nozzle at 2.0-4.0 bar atomising pressure by heating the feed to 60° C. prior to atomisation and the inlet and outlet air temperatures were 180° C. and 80° C., respectively. In an embodiment, the spray dryer has a granulation function. In an embodiment, the spray dryer is mounted with a granulation dryer.

In an embodiment, spray drying produces individual particles or agglomerates of particles.

In an embodiment, spray drying produces a mean dry particle size of about 10 μM to about 3000 μM. In an embodiment, spray drying produces a mean dry particle size of about 20 μM to about 2000 μM. In an embodiment, spray drying produces a mean dry particle size of about 10 μM to about 1000 μM. In an embodiment, spray drying produces a mean dry particle size of about 10 μM to about 500 μM.

In an embodiment, the emulsion or suspension is dried by freeze-drying to form a powder. In an embodiment, a cryoprotectant is added to the emulsion or suspension before freeze drying. In an embodiment, the cryoprotectant is a monosaccharide, disaccharide or polysaccharide, polyalcohol or a derivative thereof. In an embodiment, the cryoprotectant is selected from one or more of: trehalose, sucrose and mannitol.

In an embodiment, the emulsion or suspension is dried by drum drying to form a powder.

In an embodiment, the powder comprises about 5% to about 50% oil w/w. In an embodiment, the powder about 10% to about 50% oil w/w. In an embodiment, the powder comprises about 20% to about 50% oil w/w. In an embodiment, the powder comprises about 20% to about 50% oil w/w. In an embodiment, the powder comprises about 20% to about 40% oil w/v. In an embodiment, the powder comprises about 20% to about 30% oil w/w.

In an embodiment, the powder comprises particles of about 20 μm to about 1200 μm. In an embodiment, the powder comprises particles of about 100 μm to about 900 μm. In an embodiment, the powder comprises particles of about 400 μm to about 700 μm. In an embodiment, the powder comprises particles of about 500 μm to about 600 μm. In an embodiment, the powder comprises particles of about 1000 μm. In an embodiment, the powder is milled to further reduce the particle size. In an embodiment, milling may reduce the particle size to less than about 10 μm, or less than about 8 μm, or less than about 6 μm, or less than about 4 μm, or less than about 2 μm.

In an embodiment, the bioactive and/or bioactive precursor entrapped or encapsulated in a powder by the methods described herein is less susceptible to oxygen degradation than the same bioactive and/or bioactive precursor (e.g. oil) entrapped or encapsulated by the MicroMAX® encapsulation method (WO01/74175).

In an embodiment, the bioactive and/or bioactive precursor entrapped or encapsulated in a powder by the methods as described herein is about 500% to about 4000% more resistant to oxygen degradation than unentrapped or unencapsulated bioactive and/or bioactive precursor which time to IP is compared (see Table 7). In an embodiment, the bioactive and/or bioactive precursor entrapped or encapsulated in a powder is about 500% to about 3000% more resistant to oxygen degradation than unentrapped or unencapsulated bioactive and/or bioactive precursor which time to IP is compared. In an embodiment, the bioactive and/or bioactive precursor entrapped or encapsulated in a powder is about 500% to about 2000% more resistant to oxygen degradation than unentrapped or unencapsulated bioactive and/or bioactive precursor which time to IP is compared. In an embodiment, the bioactive and/or bioactive precursor entrapped or encapsulated in a powder is about 800% to about 2000% more resistant to oxygen degradation than unentrapped or unencapsulated bioactive and/or bioactive precursor which time to IP is compared. In an embodiment, the bioactive and/or bioactive precursor entrapped or encapsulated in a powder is about 800% to about 1500% more resistant to oxygen degradation than unentrapped or unencapsulated bioactive and/or bioactive precursor which time to IP is compared. In an embodiment, the bioactive and/or bioactive precursor entrapped or encapsulated in a powder is about 900% to about 1300% more resistant to oxygen degradation than unentrapped or unencapsulated bioactive and/or bioactive precursor which time to IP is compared.

In an embodiment, the bioactive and/or bioactive precursor entrapped or encapsulated in the powder is more resistant to oxygen degradation for at least 3 months compared to unentrapped or unencapsulated bioactive and/or bioactive precursor. In an embodiment, the bioactive and/or bioactive precursor entrapped or encapsulated in the powder is more resistant to oxygen degradation for at least 6 months compared to unentrapped or unencapsulated bioactive and/or bioactive precursor. In an embodiment, the bioactive and/or bioactive precursor entrapped or encapsulated in powder is more resistant to oxygen degradation for at least 12 months compared to unentrapped or unencapsulated bioactive and/or bioactive precursor. In an embodiment, the bioactive and/or bioactive precursor entrapped or encapsulated in the powder is more resistant to oxygen degradation for at least 18 months compared to unentrapped or unencapsulated bioactive and/or bioactive precursor. In an embodiment, the bioactive and/or bioactive precursor entrapped or encapsulated in the powder is more resistant to oxygen degradation for at least 24 months compared to unentrapped or unencapsulated bioactive and/or bioactive precursor.

In an embodiment, the bioactive and/or bioactive precursor entrapped or encapsulated in the powder is more resistant to moisture degradation for at least 3 months compared to unentrapped or unencapsulated bioactive and/or bioactive precursor. In an embodiment, the bioactive and/or bioactive precursor entrapped or encapsulated in the powder is more resistant to moisture degradation for at least 6 months compared to unentrapped or unencapsulated bioactive and/or bioactive precursor. In an embodiment, the bioactive and/or bioactive precursor entrapped or encapsulated in the powder is more resistant to moisture degradation for at least 12 months compared to unentrapped or unencapsulated bioactive and/or bioactive precursor. In an embodiment, the bioactive and/or bioactive precursor entrapped or encapsulated in the powder is more resistant to moisture degradation for at least 18 months compared to unentrapped or unencapsulated bioactive and/or bioactive precursor. In an embodiment, the bioactive and/or bioactive precursor entrapped or encapsulated in the powder is more resistant to moisture degradation for at least 24 months compared to unentrapped or unencapsulated bioactive and/or bioactive precursor.

In an embodiment, the bioactive and/or bioactive precursor entrapped or encapsulated in the powder is more resistant to pH degradation during processing compared to unentrapped or unencapsulated bioactive and/or bioactive precursor. In an embodiment, the bioactive and/or bioactive precursor entrapped or encapsulated in the powder is more resistant to pH degradation during gastrointestinal transit compared to unentrapped or unencapsulated bioactive and/or bioactive precursor.

In an embodiment, the oil encapsulated in the powder is more resistant to oxygen degradation for at least 3 months compared to unencapsulated oil. In an embodiment, the oil encapsulated in the powder is more resistant to oxygen degradation for at least 6 months compared to unencapsulated oil. In an embodiment, the oil encapsulated in the powder is more resistant to oxygen degradation for at least 12 months compared to unencapsulated oil. In an embodiment, the oil encapsulated in the powder is more resistant to oxygen degradation for at least 18 months compared to unencapsulated oil. In an embodiment, the oil encapsulated in the powder is more resistant to oxygen degradation for at least 24 months compared to unencapsulated oil.

In an embodiment, the oil encapsulated in the powder is more resistant to temperature degradation for at least 3 months compared to unencapsulated oil. In an embodiment, the oil encapsulated in the powder is more resistant to temperature degradation for at least 6 months compared to unencapsulated oil. In an embodiment, the oil encapsulated in the powder is more resistant to temperature degradation for at least 12 months compared to unencapsulated oil. In an embodiment, the oil encapsulated in the powder is more resistant to temperature degradation for at least 18 months compared to unencapsulated oil. In an embodiment, the oil encapsulated in the powder is more resistant to temperature degradation for at least 24 months compared to unencapsulated oil.

In an embodiment, the oil encapsulated in the powder is more resistant to moisture degradation for at least 3 months compared to unencapsulated oil. In an embodiment, the oil encapsulated in the powder is more resistant to moisture degradation for at least 6 months compared to unencapsulated oil. In an embodiment, the oil encapsulated in the powder is more resistant to moisture degradation for at least 12 months compared to unencapsulated oil. In an embodiment, the oil encapsulated in the powder is more resistant to moisture degradation for at least 18 months compared to unencapsulated oil. In an embodiment, the oil encapsulated in the powder is more resistant to moisture degradation for at least 24 months compared to unencapsulated oil.

In an embodiment, the oil encapsulated in the powder is more resistant to pH degradation for at least 3 months compared to unencapsulated oil. In an embodiment, the oil encapsulated in the powder is more resistant to pH degradation for at least 6 months compared to unencapsulated oil. In an embodiment, the oil encapsulated in the powder is more resistant to pH degradation during gastrointestinal transit than unencapsulated oil.

### Products

In an aspect, the present invention provides a matrix comprising protein and carbohydrate from a biomass from a first species of organism. In an embodiment, the matrix comprises one or more bioactive/s or bioactive precursor/s as described herein. In an embodiment, the matrix comprises sulforaphane. In an embodiment, the matrix comprises a glucosinolate. In an embodiment, the matrix comprises glucoraphanin.

In an aspect, the present invention provides a matrix comprising oil droplets or a bioactive and/or bioactive precursor wherein the protein and carbohydrate is from a one or more further biomasses from a further species of organism (e.g. second, third, fourth, fifth etc. species of organism).

In an aspect, the invention provides a bioactive and/or bioactive precursor entrapped or encapsulated in a matrix comprising protein and carbohydrate from a biomass from a first species of organism wherein the entrapped or encapsulated bioactive and/or bioactive precursor is more resistant to oxygen degradation when compared to the bioactive and/or bioactive precursor before entrapment or encapsulation.

In an embodiment, the bioactive and/or bioactive precursor is not from the first species of organism.

In an aspect, the invention provides a bioactive and/or bioactive precursor entrapped or encapsulated in a matrix comprising protein and carbohydrate from broccoli wherein the entrapped or encapsulated bioactive and/or bioactive precursor is more resistant to oxygen degradation when compared to the bioactive and/or bioactive precursor before entrapment or encapsulation.

In an embodiment, the bioactive and/or bioactive precursor entrapped or encapsulated in a matrix is a fatty acid. In an embodiment, the bioactive is oil.

In an embodiment, the matrix comprises protein and carbohydrate from at least one further biomass from a first species of organism.

In an embodiment, the biomass and/or further biomass comprises one or more of: i) a protein to carbohydrate ratio of between about 1:1 and about 1:10.5 ii) a protein to carbohydrate ratio of between about 1:4.5 and about 4:1; and ii) a protein to carbohydrate ratio of between about 1:2.5 and about 2:1.

In an embodiment, the biomass and/or further biomass comprises a bioactive and/or bioactive precursor.

In an embodiment, the biomass is broccoli.

In an aspect, the present invention provides an emulsion or suspension produced by the method as described herein. In an embodiment, the emulsion or suspension has an induction period, measured using the Oxipres at 80° C. and initial 5 bar oxygen pressure, of about 10 hours to about 300 hours at 80° C. In an embodiment, the emulsion or suspension has an induction period, measured using the Oxipres at 80° C. and initial 5 bar oxygen pressure, of about 100 hours to about 300 hours at 80° C.

In an aspect, the present invention provides a powder comprising an entrapped or encapsulated bioactive and/or bioactive precursor produce by the method as described herein. In an embodiment, the powder has an induction period measured using the Oxipres at 80° C. and initial 5 bar oxygen pressure, of about 10 to about 300 hours at 80° C. In an embodiment, the powder has an induction period measured using the Oxipres at 80° C. and initial 5 bar oxygen pressure, of about 50 to about 300 hours at 80° C. In an embodiment, the powder has an induction period measured using the Oxipres at 80° C. and initial 5 bar oxygen pressure, of about 80 to about 300 hours at 80° C. In an embodiment, the powder has an induction period measured using the Oxipres at 80° C. and initial 5 bar oxygen pressure, of about 100 to about 300 hours at 80° C. In an embodiment, the powder has an induction period measured using the Oxipres at 80° C. and initial 5 bar oxygen pressure, of at least 10 hours at 80° C. In an embodiment, the powder has an induction period measured using the Oxipres at 80° C. and initial 5 bar oxygen pressure, of at least 50 hours at 80° C. In an embodiment, the powder has an induction period measured using the Oxipres at 80° C. and initial 5 bar oxygen pressure, of at least 100 hours at 80° C.

In an embodiment, moisture content of the powder is about 1 to about 14%. In an embodiment, moisture content of the powder is about 1 to about 10%. In an embodiment, the moisture content of the powder is about 10% or less. In an embodiment, the moisture content of the powder is about 8% or less. In an embodiment, the moisture content of the powder is about 7% or less. In an embodiment, the moisture content of the powder is about 6% or less. In an embodiment, the moisture content of the powder is about 5% or less. In an embodiment, the moisture content of the powder is about 4% or less. In an embodiment, the moisture content of the powder is about 3% or less.

In an embodiment, the powder comprises oil. In an embodiment, the powder comprises omega-3 polyunsaturated fatty acids. In an embodiment, the powder comprises an isothiocyanate bioactive.

In an embodiment, the powder can be used as is or is a material added to or combined with other materials to from a product (e.g. a food or cosmetic product).

In an embodiment, the powder can be used to form a powder (e.g. combined with one or more other powdered ingredients), tablet, liquid, pill, capsule, or extruded product. In an embodiment, the powder is extruded. In an embodiment, the powder is compressed e.g. to form a tablet.

In an embodiment, the powder is a food, food ingredient, drink ingredient or cosmetic ingredient.

In an embodiment, the emulsion, suspension or powder can be combined with one or more other ingredients to form a product.

In an embodiment, the product is a cream, gel, tablet, liquid, pill, capsule, or extruded product.

In an embodiment, the product is a food, food ingredient, drink ingredient supplement, cosmetic or cosmetic ingredient. In an embodiment, the cosmetic is a skin hydrating product (e.g. moisturizer or face mask).

In an embodiment, the product comprises omega-3 polyunsaturated fatty acids.

In an embodiment, the food is an animal feed. In an embodiment, the animal feed comprises an omega-3 polyunsaturated fatty acid. In an embodiment, the omega-3 polyunsaturated fatty acid is selected from one or more of: a-linolenic acid (ALA), eicosapentaenoic acid (EPA), and docosahexaenoic acid (DHA) and docosapentaenoic acid (DPA). In an embodiment, the animal feed comprises astaxanthin and/or a-lipoic acid.

In an embodiment, wherein the animal feed is an aquaculture feed.

In an embodiment, the product is a food ingredient for e.g. infant formulae, children formula, adult formula, yoghurts, beverages, elderly supplement, ultra-high temperature processed (UHT) drinks (e.g. milk), soup, dips, pasta products, bread, snacks and other bakery products processed cheese, and/or animal feed (including aquaculture feed). In an embodiment, bioactive and/or bioactive precursor encapsulated or entrapped in the food ingredient is more stable when added to a product than unencapsulated or unentrapped bioactive and/or bioactive precursor.

In an embodiment, the product is suitable for use as a cosmetic or cosmetic ingredient, for example, as a lipstick, cream, lotion, or ointment.

In an embodiment, the product is a powdered supplement. In an embodiment, the powdered supplement is dissolved in water or added to a food, beverage or a meal.

In an embodiment, the product is an emulsion or suspension or powder.

### Preparation of a Pharmaceutical Composition

In an aspect, the present invention provides a method of producing an emulsion comprising an isothiocyanate and/or isothiocyanate precursor, the method comprising: providing a mixture comprising water, a lipid, and an isothiocyanate and/or isothiocyanate precursor, thereby forming an emulsion. Such emulsions are suitable for use in pharmaceutical compositions.

In an embodiment, the mixture comprising water and isothiocyanate and/or isothiocyanate precursor is admixed with a lipid. In an embodiment, the aqueous suspension comprising isothiocyanate and/or isothiocyanate precursor, and comprising protein and/or carbohydrate, is admixed with a lipid. In an embodiment, the isothiocyanate and/or isothiocyanate precursor is admixed with a lipid, and the resulting composition is admixed with an aqueous medium. In an embodiment, the aqueous medium comprises protein and/or carbohydrate. In an embodiment, the isothiocyanate and/or isothiocyanate precursor is admixed with a mixture comprising water and a lipid. In an embodiment, the isothiocyanate and/or isothiocyanate precursor is admixed with a mixture comprising water and a lipid. In an embodiment, the protein or carbohydrate is from the same single species of organism. In an embodiment, the protein, carbohydrate or isothiocyanate and/or isothiocyanate precursor is form the same single species of organism.

In an embodiment, following about one month of storage at about 4 to about 10° C. or at about −18° C., the concentration of the isothiocyanate and/or isothiocyanate precursor in the emulsion is at least twice the concentration of isothiocyanate and/or isothiocyanate precursor in a corresponding composition lacking the lipid. In an embodiment, following about one month of storage at about 4 to about 10° C. or at about −18° C., the concentration of the isothiocyanate and/or isothiocyanate precursor in the emulsion is at least three times the concentration of isothiocyanate and/or isothiocyanate precursor in a corresponding composition lacking the lipid. In an embodiment, following about two months of storage at about 4 to about 10° C. or at about −18° C., the concentration of the isothiocyanate and/or isothiocyanate precursor in the emulsion is at least twice the concentration of isothiocyanate and/or isothiocyanate precursor in a corresponding composition lacking the lipid.

In an aspect, the present invention provides a method of preparing a powder comprising an isothiocyanate and/or isothiocyanate precursor, comprising: preparing an emulsion as described therein, and subjecting the emulsion to drying conditions, thereby removing water. In an embodiment, the emulsion is subjected to freeze-drying or spray drying conditions, thereby forming a powder.

In an embodiment, following about one month of storage at −18° C., the concentration of the isothiocyanate and/or isothiocyanate precursor in the powder is at least one times the concentration of isothiocyanate and/or isothiocyanate precursor in a corresponding powder lacking the lipid.

In an embodiment, following about one month of storage at −18° C., the concentration of the isothiocyanate and/or isothiocyanate precursor in the powder is at least twice the concentration of isothiocyanate and/or isothiocyanate precursor in a corresponding powder lacking the lipid.

In an embodiment, following about two months of storage at-18° C., the concentration of the isothiocyanate and/or isothiocyanate precursor in the powder is at least twice the concentration of isothiocyanate and/or isothiocyanate precursor in a corresponding powder lacking the lipid.

In an aspect, the present invention provides a method of preparing a pharmaceutical or cosmetic composition, comprising: preparing an emulsion as described herein, or preparing a powder as described herein, and converting the emulsion or dried composition to a pharmaceutical or cosmetic composition.

### Pharmaceutical Compositions

In an embodiment, the present invention provides a pharmaceutical or cosmetic composition produced by the methods or from the emulsion or powder described herein comprising an isothiocyanate and/or an isothiocyanate precursor, a lipid, and a pharmaceutical and/or cosmetic excipient.

In an aspect, the present invention provides a pharmaceutical or cosmetic composition, which comprises an isothiocyanate and/or an isothiocyanate precursor, a lipid, and a pharmaceutical and/or cosmetic excipient. In an embodiment, the pharmaceutical or cosmetic composition further comprises protein and/or carbohydrate.

In an aspect, the present invention provides an emulsion comprising water, a lipid, and an isothiocyanate and/or isothiocyanate precursor. Such emulsions are suitable for use in pharmaceutical and cosmetic compositions.

In an embodiment, the composition is for topical, enteral/gastrointestinal or parenteral administration. In an embodiment, includes application to a localized area of the skin and also includes transdermal administration (administration via absorption through the skin). In an embodiment, enteral/gastrointestinal includes, for example, oral, rectal, stomach, gastrointestinal tracht, sublabial, buccal, sublingual. In an embodiment, paraenteral includes, for example, transdermal, intramuscular and in intravenous. In an embodiment, the composition is in the form of a cream, ointment, gel, tablet, liquid, pill, capsule, powder or extruded product.

In an embodiment, following storage for a period of about one month, about 10 to about 90% of the isothiocyanate and/or isothiocyanate precursor remain in the composition. In an embodiment, following storage for a period of about one month, at least 10% of the isothiocyanate and/or isothiocyanate precursor remain in the composition. In an embodiment, following storage for a period of about one month, at least 20% of the isothiocyanate and/or isothiocyanate precursor remain in the composition. In an embodiment, following storage for a period of about one month, at least 30% of the isothiocyanate and/or isothiocyanate precursor remain in the composition. In an embodiment, following storage for a period of about one month, at least 40% of the isothiocyanate and/or isothiocyanate precursor remain in the composition. In an embodiment, following storage for a period of about one month, at least 50% of the isothiocyanate and/or isothiocyanate precursor remain in the composition.

In an embodiment, the isothiocyanate is selected from one or more of: sulforaphane, allyl isothiocyanate, benzyl isothiocyanate and phenethyl isothiocyanate.

In an embodiment, the isothiocyanate precursor is selected from one or more of: glucosinolate, glucoraphanin, sinigrin, glucotropaeolin, and gluconasturtiin.

In an embodiment, the lipid is an oil as described herein. In a preferred embodiment, the oil is selected from canola oil, olive oil, sunflower oil, fish oil or an algal oil. In an embodiment, the composition comprises about 10% to about 90% oil. In an embodiment, the composition comprises about 20% to about 80% oil. In an embodiment, the composition comprises about 30% to about 70% oil. In an embodiment, the lipid is a wax as described herein.

In an embodiment, the emulsion or powder is combined with one or more excipients, carriers or additives which should be pharmaceutically or cosmetically acceptable in the sense of being compatible with the other ingredients of the formulation and not unduly deleterious to the recipient thereof, which could include e.g., polyvinylpyrrolidones, derivatised celluloses such as hydroxymethylcellulose, hydroxyethylcellulose, and hydroxypropylmethylcellulose, ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients, carriers and/or additives suitable for use in the compositions are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

In an aspect, the present invention provides a method of therapy or prophylaxis of a condition, comprising administering to a subject in need thereof an effective amount of a pharmaceutical composition, emulsion or powder as described herein. In an aspect, the present invention provides a pharmaceutical composition, emulsion or powder as described herein, for use in therapy or prophylaxis of a condition. In an aspect, the present invention provides a method of treating or preventing a condition in a subject, comprising administering to the subject an effective amount of a pharmaceutical composition, emulsion or powder as described herein. Use of a pharmaceutical composition as described herein in the manufacture of a medicament for the treatment or prevention of a condition. Use of an emulsion as described herein or a powder as described herein for the manufacture of a medicament for the treatment or prevention of a condition.

In an aspect, the present invention provides a method of therapy or prophylaxis, use or method of treating or preventing as described herein, wherein the condition is selected from: cancer, diabetes, cardiovascular disease, autism, osteoporosis, neuroprotective diseases, metabolic syndrome, inflammation, oxidative stress and a gut health condition. In an embodiment, the gut health condition is selected from ulcerative colitis, irritable bowel syndrome, Crohn's, small intestinal overgrowth, leaky gut and lactose intolerance. In an embodiment, the condition is cancer.

### EXAMPLES

### Example 1—Demonstration of the Emulsifying and Physical Functionality of Broccoli as an Encapsulant to Prepare Physically Stable Oil in Water Emulsion

Raw broccoli was blended with added water (1:1 ratio). To this was added an oil such that the ratio of broccoli:water:oil was 1:1:1 and the whole mixture was blended using a benchtop blender. The oil-in-water emulsion was physically stable over 2 h. While blended oil-water mixture without the broccoli immediately separated into 2 phases as expected

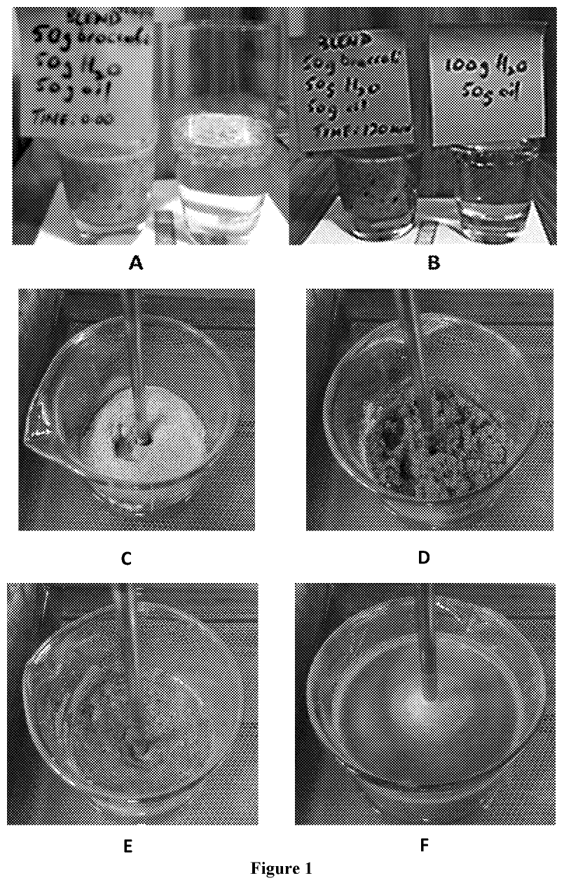

(FIG. 1A-B).

### Example 2—Preparation of Aqueous Phase Suspension Using Freeze Dried Broccoli Powder as Encapsulant

Freeze dried broccoli powder was put into a beaker, water (60° C.) was added while mixing using an overhead mixer, until a flowable mixture was achieved (7.46% total solids (TS)) (FIG. 1C-F). The pH of the mixture was adjusted from 6.01 to 7.50 using 2N NaOH. Then the mixture was heat treated at 75° C. for 2 min or at 100° C. for 30 min then cooled down to 60° C.

### Example 3—Preparation of Aqueous Phase Suspension Using Raw Broccoli as Encapsulant

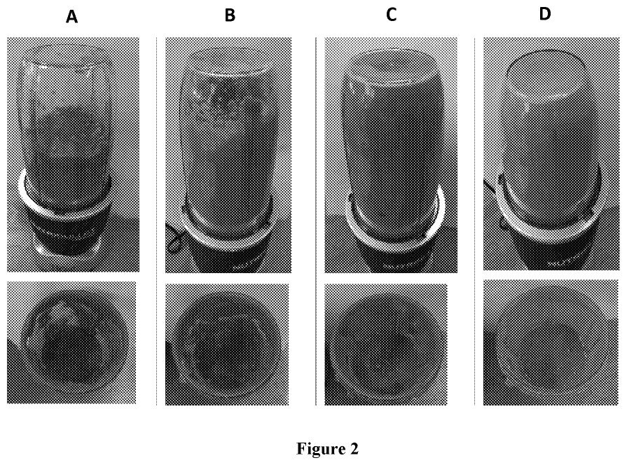

Raw broccoli (10% TS) was cut into small pieces, boiling water was added initially and the mixture was blended to obtain broccoli suspension with different total solids (FIG. 2A: 7.66% (TS), (FIG. 2B: 6.87% TS), (FIG. 2C: 6.23% TS), (FIG. 2D: 4.99% TS). From this initial experiment the 4.99% TS mixture was chosen for preparation of encapsulant. The pH of this mixture was adjusted from 6.23 to 7.50 using 2N NaOH. Then the mixture was heat treated at 75° C. for 2 min or at 100° C. for 30 min then cooled down to 60° C.

### Example 4—Demonstration of Freeze Dried Broccoli Powder for Encapsulation of Omega-3 Oil in Emulsions and Powder Format

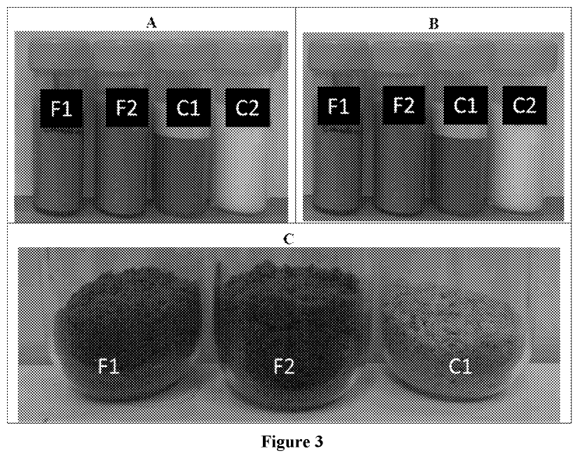

The aqueous phase suspensions (5% TS) made from freeze dried broccoli powders described in Example 2 were used as encapsulant. Tuna oil was added (1:1 broccoli solids:oil ratio) into the aqueous phase suspension (60° C.) described in Example 2, and homogenised using an Ultra Turrax at 15,000 rpm for 3 min to prepare the emulsions F1 heated at 75° C. for 2 min, and F2 heated at 100° C. for 30 min (FIG. 3A).

Aqueous phase preparation using 1 Sodium caseinate (NaCas)-1 glucose (Glu)-1 dried glucose syrup (DGS) solution was prepared in 60° C. water for 40 min (25% TS), pH was adjusted to 7.50 and heat treated at 100° C. for 30 min and cooled down to 60° C. (as control, C1), Tween 80 (0.5 g) was stirred to 87 g 60° C. water for 5 min as another control using low molecular weight emulsifier (C2).

Omega-3 oil in water emulsions were made by combining oil with protein (Na-Caseinate) and carbohydrates (glucose and dried glucose syrup) C1, or a low molecular weight emulsifier C2, were also prepared for comparison (FIG. 3A).

F1, F2, C1 and C2 emulsions were all physically stable after overnight storage at 20° C. (FIG. 3B). F1, F2 and Cl were also freeze dried to obtain an omega-3 oil powder containing 50% omega-3 oil (FIG. 3C).

### Example 5—Oxidative Stability of Omega-3 Oil Emulsion and Powders Tested Under Accelerated Oxidation

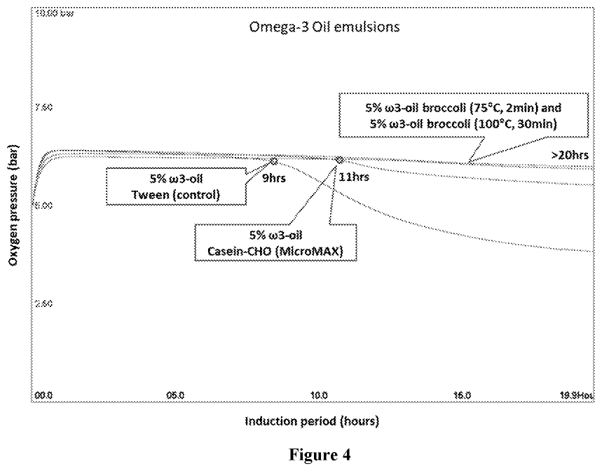

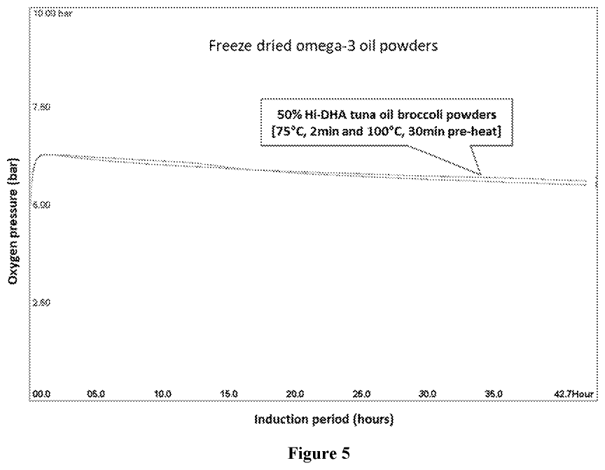

Emulsions (F1, F2, C1, C2) and freeze dried powders (F1, F2) described in Example 4 were tested under accelerated oxidation at 80° C. under initial 5 bar oxygen pressure using an Oxipres apparatus (Mikrolab Aarhus A/S, Højbjerg, Denmark). Fish oil emulsions and powders made with broccoli matrix (F1 and F2) took up oxygen slower that corresponding fish oil emulsions (F1 and F2) and emulsion (C1) encapsulated using CSIRO's MicroMAX® technology and fish oil emulsions made with Tween (FIGS. 4 and 5 and Table 2).

TABLE 2
Oxipres results of omega-3 oil emulsions-broccoli compared to Na-
Caseinate-dried glucose syrup (DGS)-glucose (Glu) as encapsulant or low
molecular weight emulsifier (Tween 80).

| Encapsulant / | Heat | | | Slope |
|---|---|---|---|---|
| Emulsifier | Treatment | ^{#}Sample | IP (hr) at 80° C. | (-mBar/hr) |
| F1 Broccoli | 75° C., 2 min | Emulsion | >20.0 | n/a |
| Powder | | | | |
| F2 Broccoli | 100° C., 30 min | Emulsion | >20.0 | n/a |
| Powder | | | | |
| F3 (1NaCas- | 100° C., 30 min | Emulsion | 11 | −152 |
| 1DGS-1Glu) | | | | |
| F4 Tween 80 | 75° C., 2 min | Emulsion | 9 | −391 |
| F1 Broccoli | 75° C., 2 min | FD Powder | >43.0 | n/a |
| Powder | | | | |
| F2 Broccoli | 100° C., 30 min | FD Powder | >43.0 | n/a |
| Powder | | | | |

“>” is used when IP is not distinctive when tested; ^{#}Emulsion (9.5% TS, 4.8% oil), powder (50% oil); (8 g powder , 4 g oil tested); n/a—not applicable

### Example 6—Demonstration of Using Broccoli Powder for Stabilisation Omega-3 Oil in Powder

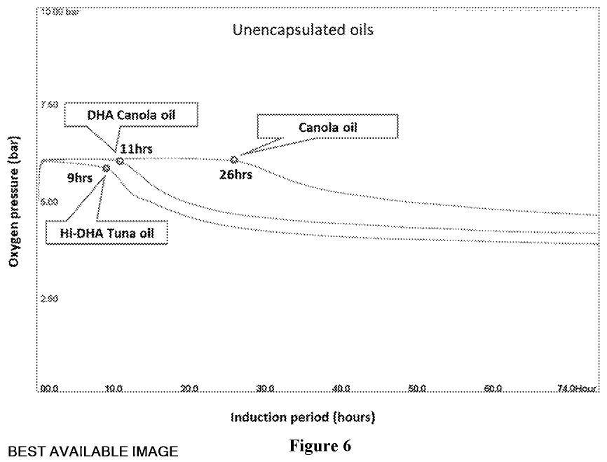

The Oxipres data for the neat oils are given in FIG. 6. The effect of the amount of encapsulant matrix on oxygen uptake was assessed by Oxipres test on broccoli matrix (without oil). The results are given in FIG. 7.

Broccoli heads was cut into quarter and macerated by adding water (5% TS), heated to 79° C., 4 min and freeze dried. Freeze dried broccoli powder was reconstituted in water (5% TS) and used for encapsulation. Oil (tuna oil, DHA canola oil or canola oil) was added into the suspension to get a 12.5%, 25% and 50% oil powder, and homogenised using an Ultraturrax at 15,000 rpm for 3 min to prepare the emulsions. The emulsions (5.7%, 6.6% and 9.5% total solids respectively) were freeze dried, and tested under accelerated oxidation conditions using an Oxipres unit at 80° C. with initial oxygen press 5 bar in room temperature.

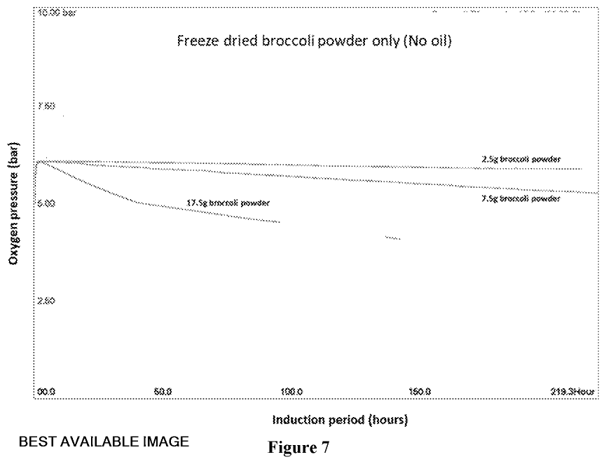

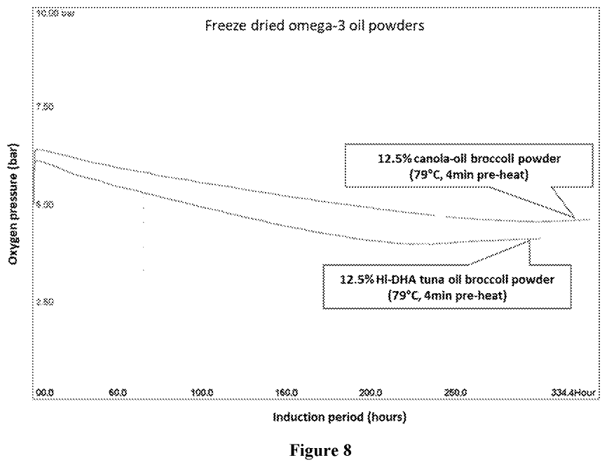

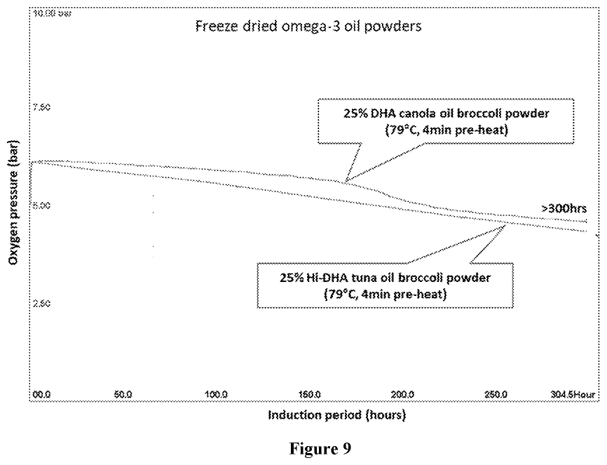

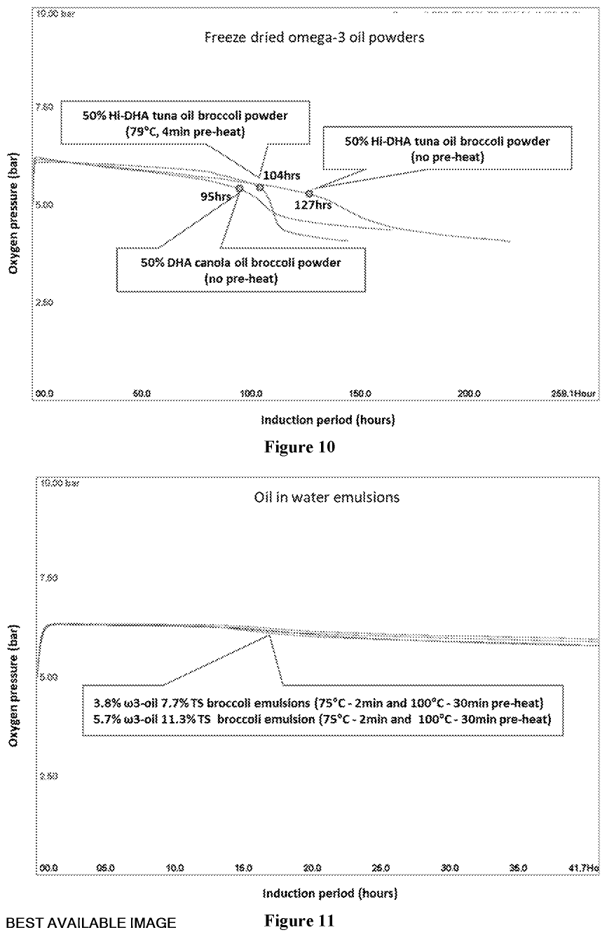

Results are shown in FIGS. 8 to 10. Slow oxygen uptake in these samples is in part due to the oxygen uptake by the broccoli matrix. No clear IP observed in 12.5% and 25% oil powders up to 300 h.

### Example 7—Demonstration of Using Raw Broccoli for Preparation and Stabilisation of Omega-3 Oil in Water Emulsion

The aqueous phase suspensions (5% & 6% TS) made from raw broccoli described in Example 3 were used as encapsulant. Omega-3 oil was added (1:1 broccoli solids:oil ratio) into the aqueous phase suspension (60° C.) described in Example 3, and homogenised using an Ultraturrax at 15,000 rpm for 3 min to prepare the emulsions E1 and E2 heated at 75° C. for 2 min, and E3 and E4 heated at 100° C. for 30 min. Final emulsion to total solids were 7.7% and 11.3% respectively.

Emulsions were tested under accelerated oxidation conditions using an Oxipres unit at 80° C. with initial oxygen press 5 bar in room temperature. Results are shown in FIG. 11 and Table 3. Both heated and unheated broccoli performed in the same way with respect to oxygen uptake.

TABLE 3
Oxidative stability (Oxipres results) of omega-3 oil emulsions
(5-6% oil w/w) using broccoli as encapsulants.

| | Emulsion | | | Omega-3 oil Emulsion | |
|---|---|---|---|---|---|
| | Total | Viscosity | Heat | IP (hr) at | Slope |
| Encapsulant | Solids | (cP) | treatment | 80° C. | (-mBar/hr) |
| Emulsion 1 | 9.5% | 1451 | 75° C., 2 | >42.0 | n/a |
| Broccoli | | | min | | |
| Emulsion 2 | 11.3% | 2682 | 75° C., 2 | >42.0 | n/a |
| Broccoli | | | min | | |
| Emulsion 3 | 9.5% | 2130 | 100° C., | >42.0 | n/a |
| Broccoli | | | 30 min | | |
| Emulsion 4 | 11.3% | 4653 | 100° C., | >42.0 | n/a |
| Broccoli | | | 30 min | | |

“>” is used when IP is not distinctive when tested; n/a —not applicable

### Example 8—Oxipres Results on Raw Broccoli-Tuna Oil Freeze Dried Powders

Raw broccoli (5% and 6% TS) was blended with boiling water at high speed for 3 min (temperature 68.3° C. measured). The pH was adjusted to 7.5. One half was pasturised at 75° C. for 2 min and another half was heat treated at 100° C. for 30 min. Both of them were cooled to 60° C. and tuna oil was added and ultra-turraxed for 2 min at 15,000 rpm. The resultant emulsions containing 9.5% and 11.3% TS respectively were then freeze dried. Emulsion formulations (9.5% total solids) using freeze dried broccoli and using Na-caseinate and carbohydrates as encapsulant were also prepared and freeze dried and tested for comparison.

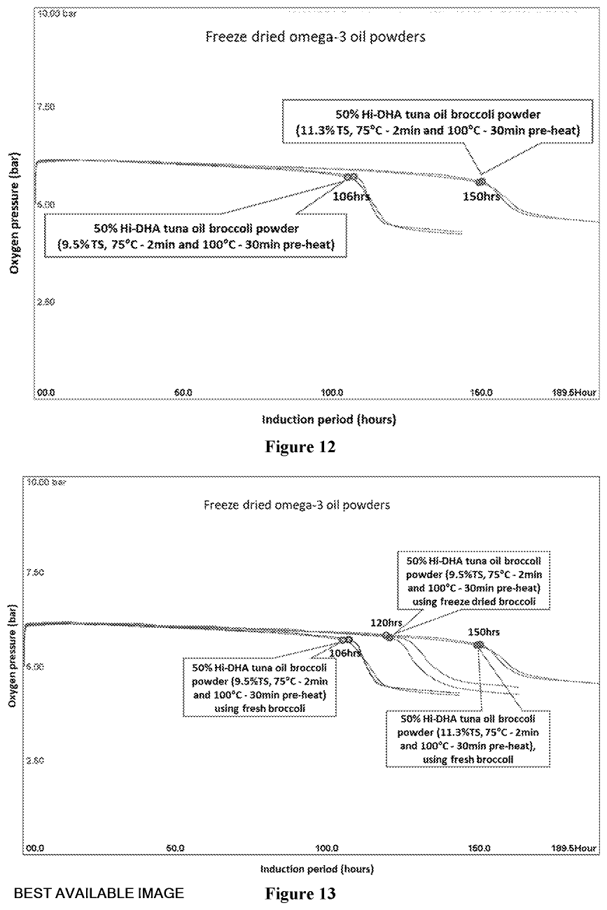

Freeze dried emulsions were tested using Oxipres unit at 80° C. with initial oxygen press 5 bar in room temperature. Results are shown in FIG. 12, FIG. 13 and Table 4.

TABLE 4
Oxidative stability (Oxipres results) of omega-3 oil powders (50% oil
w/w) using raw broccoli as encapsulants.

| | Emulsion | | Omega-3 oil Powders | |
|---|---|---|---|---|
| | Total | | IP (hr) at | Slope |
| Encapsulant | Solids | Heat treatment | 80° C. | (-mBar/hr) |
| P1 Broccoli (Fresh) | 9.5% | 75° C., 2 min | 105.1 | −108 |
| P2 Broccoli (Fresh) | 11.3% | 75° C., 2 min | 149.4 | −71 |
| P3 Broccoli (Fresh) | 9.5% | 100° C., 30 min | 107.1 | −192 |
| P4 Broccoli (Fresh) | 11.3 % | 100° C., 30 min | 150.4 | −81 |
| P4 Broccoli (Freeze | 9.5% | 75° C., 2 min | 119.4 | −91 |
| dried) | | | | |
| P5 Broccoli (Freeze | 9.5% | 100° C., 30 min | 120.4 | −127 |
| dried) | | | | |

50% oil powder (8 g powder, 4 g oil tested)

### Example 9—Oxygen Uptake of Omega-3 Broccoli Emulsion Samples Made Using Pre-Processed Broccoli as Encapsulant

The oxygen uptake of broccoli emulsion using variously pre-processed broccoli (fresh broccoli, steamed, steamed broccoli, steamed shredded broccoli an steamed pureed drum dried broccoli) followed by drum drying was assessed by_Oxipres test at 80° C. with initial 5 bar oxygen pressure.

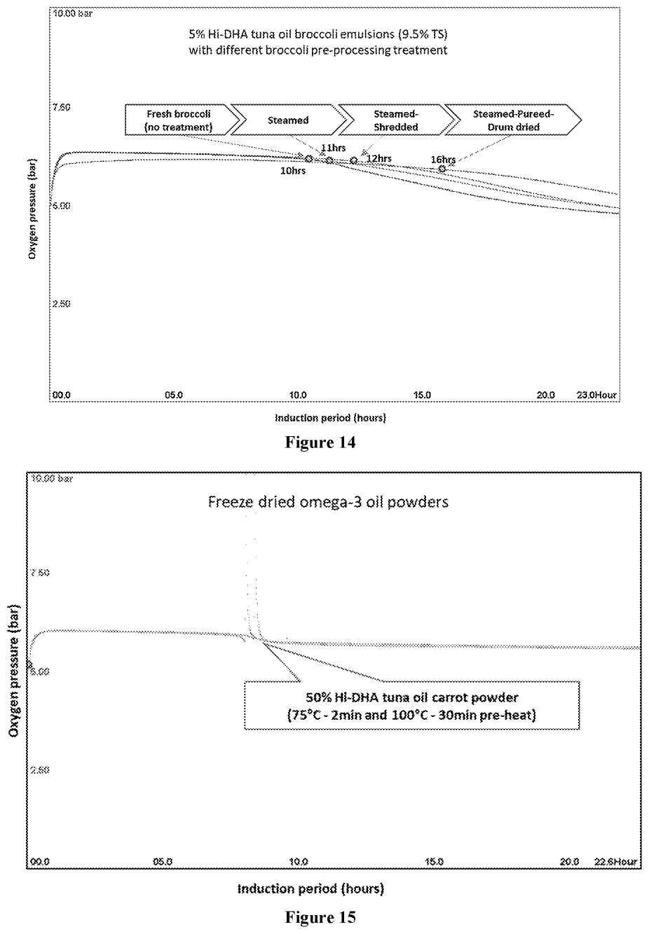

Results are shown in FIG. 14. The broccoli encapsulant was used at different stages of processing and made up to 5% aqueous solids. The emulsions were prepared at 9.5% TS and 4.8% oil. The IP (h) is where there is a significant increase in oxygen uptake (sharp decline in oxygen pressure). Sample tested contained 4 g oil and 4 g matrix. The slow oxygen uptake in these samples is in part due to the oxygen uptake by the broccoli matrix.

### Example 10—Biomasses as Encapsulants

Raw biomass was cut into small pieces, boiling water was added initially and the mixture was blended to obtain an aqueous suspension containing the biomass (5% TS). The pH of this mixture was adjusted to 7.50 using 2N NaOH. Then the mixture was heat treated at 75° C. for 2 min or at 100° C. for 30 min then cooled down to 60° C. Omega-3 oil was added (1:1 biomass solids:oil ratio) into the aqueous phase suspension (60° C.), and homogenised using an Ultraturrax at 15,000 rpm for 3 min to prepare the emulsions which were freeze dried to obtain powders (50% oil).

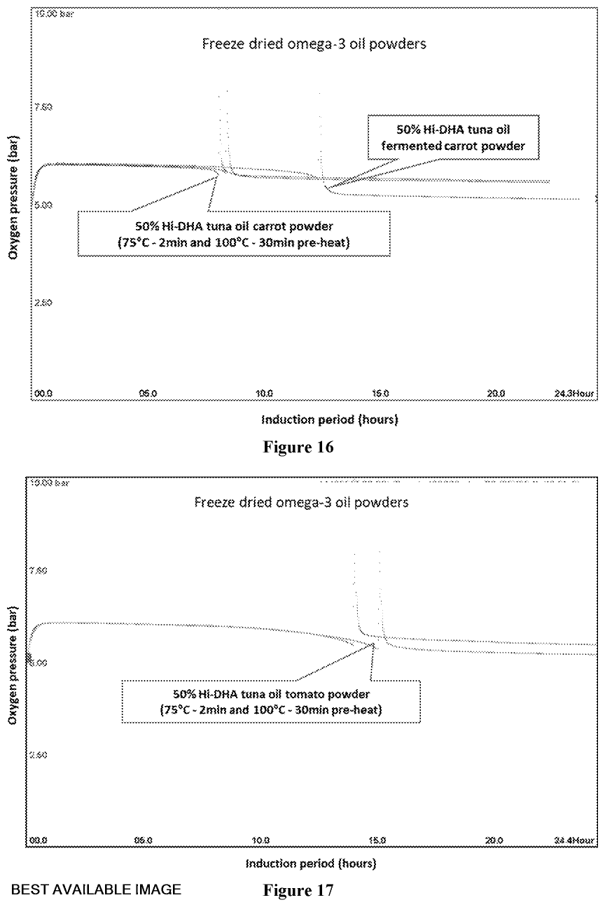

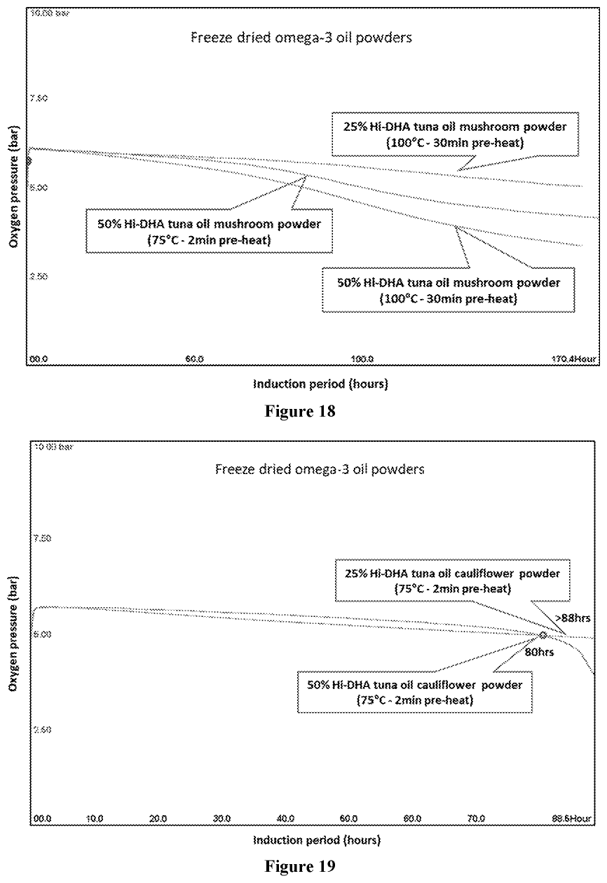

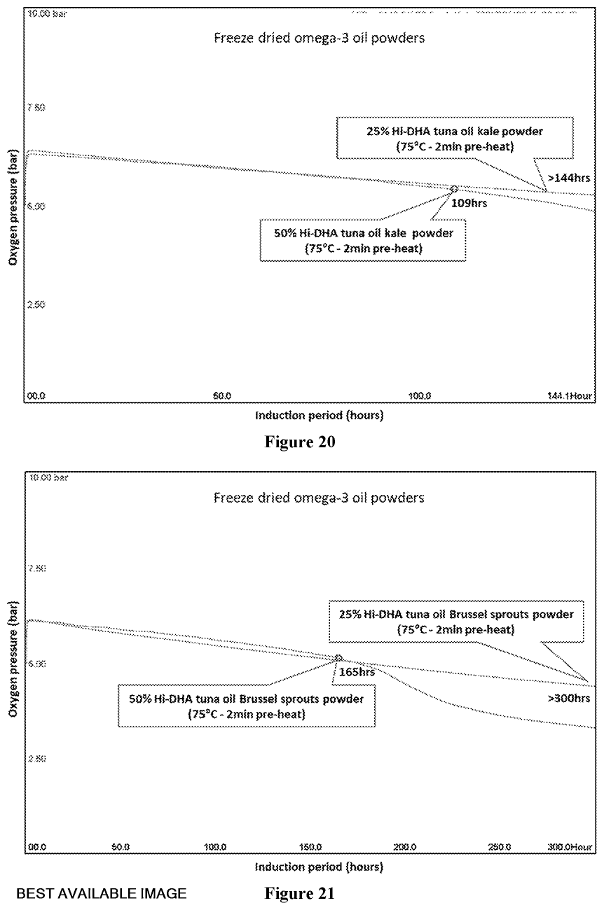

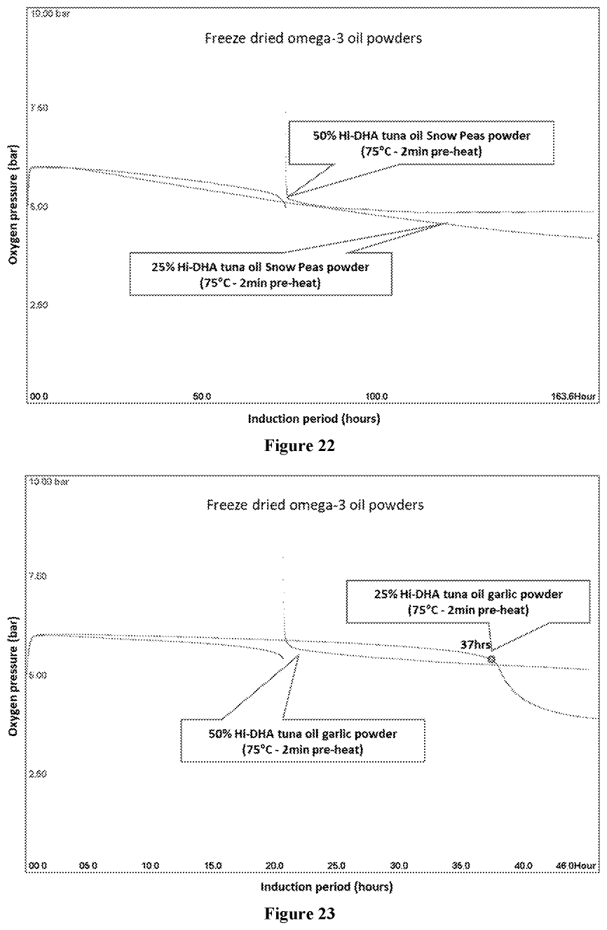

The results are provided in Table 5 and FIGS. 15 to 23. These results show the relative oxidative stability of the oils, with the longer induction period being associated with greater protection afforded by the encapsulant to the oil under accelerated conditions.

TABLE 5
Oxidative stability of omega-3 oil powders
(25% and 50% oil content).

| | | | | 25% Oil Powder | |
|---|---|---|---|---|---|
| | | 50% Oil Powder | | | Slope |
| Encap- | | IP (hr) at | Slope | IP (hr) at | (-mBar/ |
| sulant | Treatment | 80° C. | (-mBar/hr) | 80° C. | hr) |
| Carrot | 75° C., 2 | >20 | **, n/a | na | |
| | min | | | | |
| Carrot | 100° C., 30 | >20 | **, n/a | | |
| | min | | | | |
| Carrot | Fermented | >20 | **, n/a | | |
| powder | | | | | |
| Tomato | 75° C., 2 min | >20 | **, n/a | | |
| Tomato | 100° C., 30 | >20 | **, n/a | | |
| | min | | | | |
| Mushroom | 75° C., 2 min | >170 | n/a | | |
| Mushroom | 100° C., 30 | >170 | n/a | >170 | n/a |
| | min | | | | |
| Cauliflower | 75° C., 2 min | 80.5 | −237 | >88 | n/a |
| Kale | 75° C., 2 min | >100 | n/a | >144 | n/a |
| Brussel | 75° C., 2 min | 165 | −25 | >300 | n/a |
| sprouts | | | | | |
| Snow Peas | 75° C., 2 min | >160 | **, n/a | >160 | n/a |
| Garlic | 75° C., 2 min | >46 | **, n/a | 38 | −2640 |

**Sudden increase in pressure at IP leading to release of volatiles, n/a—not applicable as not possible to obtain rate of oxygen uptake due to lack of distinctive IP; 50% oil powder (8 g powder, 4 g oil tested), 25% oil powder (12 g powder, 3 g oil tested); na-not tested

### Example 11—Effect of Added Protein to Biomass as Encapsulant

Raw carrot was cut into small pieces, added into boiling water and blended to obtain an aqueous suspension containing the biomass (5% TS). The pH of this mixture was adjusted to 7.50 using 2N NaOH. Then the mixture was heat treated at 90° C. for 5 min then cooled down to 60° C. Different protein dispersion (10% TS) (Nacaseinate, soy protein isolate, pea protein) was added to the carrot suspension to obtain a 1:2 protein:CHO ratio. Tuna oil was added to the carrot-protein mixture (60° C.), and homogenised using an Ultraturrax at 15,000 rpm for 3 min to prepare the emulsions which were freeze dried to obtain powders (25% oil).

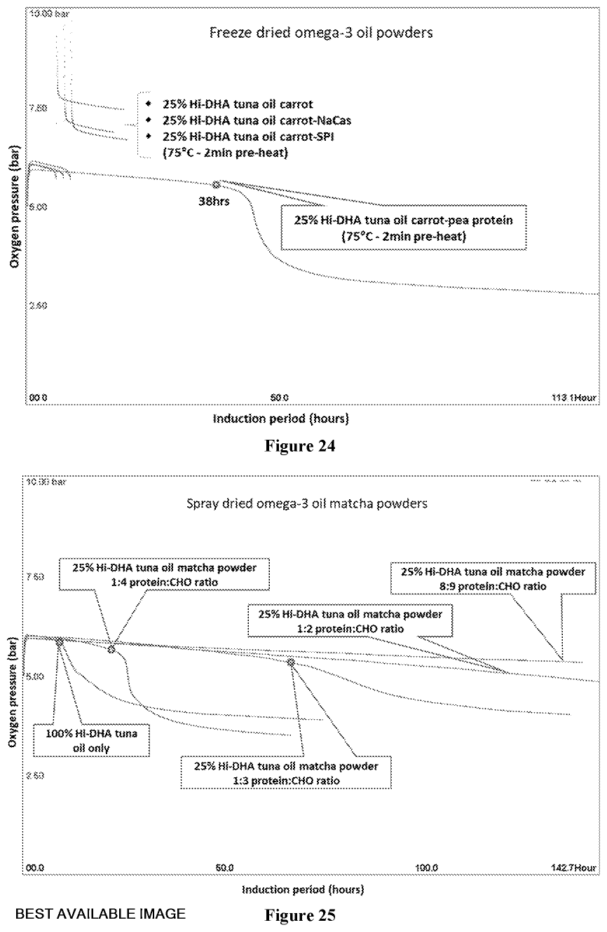

The results are provided in FIG. 24. Only the carrot-pea protein omega-3 powders had a clear IP (38 h). With all other samples, there was a sudden increase in pressure during the Oxipres test.

### Example 12—Matcha as Encapsulant with and without Added Carbohydrate

Matcha powder (with or without added maltodextrin) was reconstituted in water (45° C., 1 h). The protein to carbohydrate (CHO) ratio (w/w) of in different formulation were 8:9 (matcha only), 1:2, 1:3 and 1:4 for formulations with added maltodextrin. Tuna oil was added and dispersed using a Silverson mixer (Silverson L4R, Silverson Machines Ltd., Chesham, Buckinghamshire, UK) for 3 min, and emulsions were homogenized at 250/100 bar (Avestin Emulsiflex C5, Avestin Inc., Ottawa, Ontario, Canada). The emulsions (15% total solids) were spray dried to obtain powders containing 25% fish oil (dry basis).

The Oxipres test at 80° C. with initial 5 bar oxygen pressure is shown in FIG. 25. The slow oxygen uptake in these samples with 1:2 and 8:9 protein carbohydrate ratio is in part due to the oxygen uptake by the matrix. Clear IP is shown for tuna oil only, and samples with 1:4 and 1:3 protein carbohydrate ratio.

### Example 13—Oxipres Result Showing Stability of Spray Dried Omega-3 Oil Powder Comparing “Freeze Dried Broccoli” Powder and “Na-Caseinate+Carbohydrate” as Encapsulant

Freeze dried broccoli powder was reconstituted in 60° C. water to 5% TS, and allowed to hydrate for 1 hr. Tuna oil heated to 60° C. was added and homogenised using a Silverson emulsifier for 5 minutes at maximum speed. The emulsions were then spray dried. For comparison a MicroMAX® formulation using heated NaCaseinate-glucose-dried glucose sugar solution heated at 100° C. for 30 minutes was used as encapsulant. The same oil was added and homogenised at 180/80 bar pressure at 60° C. Both formulations were spray dried in a lab-scale Drytec Spray dryer using a twin fluid nozzle (4 bar pressure). Spray dried powders were tested using Oxipres unit at 80° C. with initial oxygen press 5 bar in room temperature.

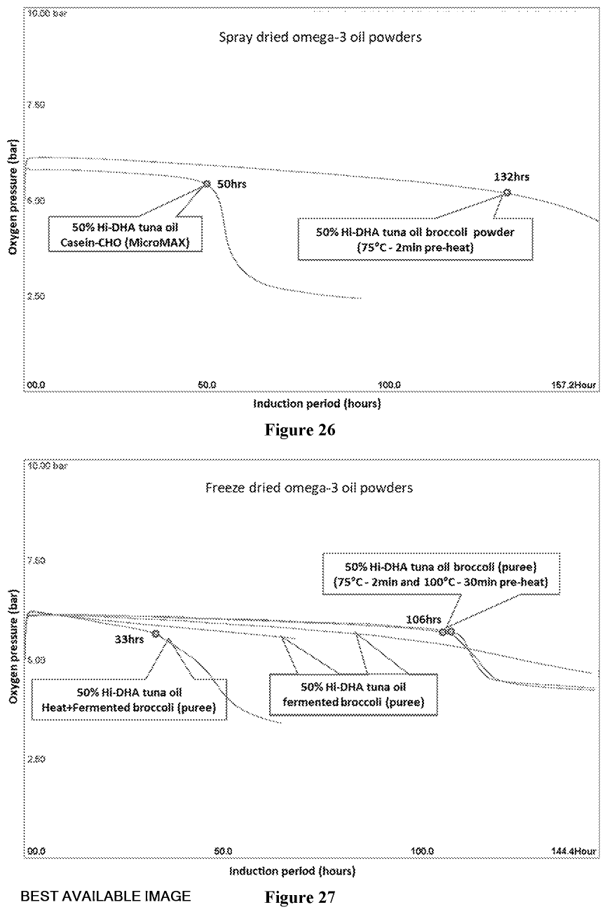

Oxipres test results (FIG. 26) show the induction period (IP) of spray dried (50% tuna oil) omega-3 broccoli powder, tested at 80° C. with initial 5 bar oxygen pressure compared to that of 50% tuna oil powder using heated casein-carbohydrate as encapsulant. There is a clear IP for both samples. The results show that oil encapsulated via the methods as described herein is more resistant to oxygen degradation than oil encapsulated using MicroMAX® technology.

### Example 14—Comparison Between Using Fermented and Non-Fermented Broccoli as Encapsulant for Production of Omega-3 Oil Broccoli Powders

Broccoli puree (heated at 75° C., 2 min or 100° C. 30 min) or fermented broccoli puree (±heat treatment before fermentation) was prepared (5% TS). Hi-DHA tuna oil was added and homogenised using a Silverson emulsifier-mixer (60° C. for 5 minutes) to form the emulsion. All formulations were freeze dried. The powders were tested in an Oxipres at 80° C. with initial 5 bar oxygen pressure and results are shown in FIG. 27. There is a clear IP for samples using non-fermented broccoli powder, but no clear IP for sample using fermented broccoli puree.

### Example 15—Comparison of Stability of EPA and DHA in Tuna Oil Powders Against Unencapsulated Tuna Oil

Selected powders from Examples 5 and 8 and neat tuna oil (unencapsulated) were stored in lightly capped bottles in a 40° C. oven. The fatty acid analysis of the oil and oil in powder samples measured using gas chromatography. The eicosapentaenoic acid (EPA) and docosahexaenoic acid (DHA) content of the initial tuna oil and those in the stored samples are given in Table 6. The higher content of EPA and DHA in stored powders compared to EPA and DHA in the neat oil after 1 month 40° C. clearly demonstrates that encapsulant protection of EPA and DHA against oxidation.

TABLE 6
EPA and DHA content of initial oil and that of stored tuna oil
and powders (1 month at 40° C.).

| | Heat | | | |
|---|---|---|---|---|
| | Treatment of | | EPA | DHA |
| Encapsulant | Encapsulant | Sample | % in oil | % in oil |
| Unencapsulated Tuna Oil | Not applicable | Initial Oil | 5.8 | 28.8 |
| | | Stored Oil | 4.0 | 17.3 |
| F1 Broccoli Powder | 75° C., 2 min | Stored FD | 5.3 | 26.6 |
| | | Powder | | |
| F2 Broccoli Powder | 100° C., 30 min | Stored FD | 5.3 | 26.4 |
| | | Powder | | |
| F3 (1NaCas-1DGS-1Glu) | 100° C., 50 min | Stored FD | 5.0 | 24.3 |
| | | Powder | | |
| P1 Broccoli (raw) | 75° C., 2 min | Stored FD | 5.5 | 27.6 |
| | | Powder | | |
| P3 Broccoli (raw) | 100° C., 30 min | Stored FD | 5.4 | 27.4 |
| | | Powder | | |

Storage: l g powder in sealed glass vials stored at 40° C .

### Example 16—Headspace Analysis of Secondary Oxidation Products from Omega-3 Oil Powders Using Selected Biomasses as Encapsulants

Freeze dried omega-3 oil powders from example 8 and 10 were stored at −18° C. for 14 months, and selected powders from these examples were sampled and stored in lightly capped bottles in a 40° C. oven for 4 weeks for analysis. The analysis of headspace secondary oxidation products was carried out using GC-MS.

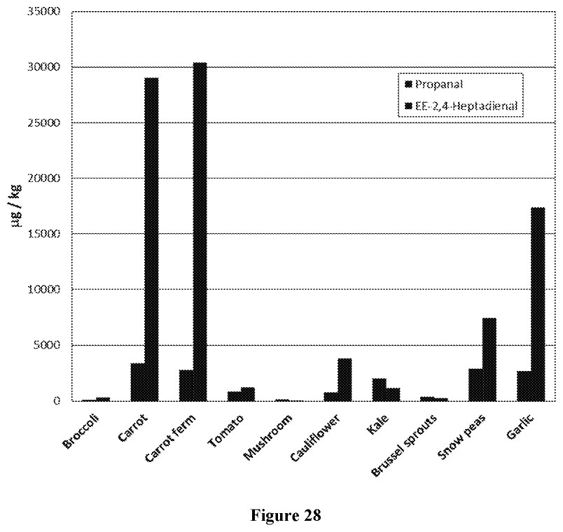

The results are provided in FIG. 28. The lower the amount of propanal and EE-2,4-Heptadienal means that the sample is more stable to oxidation. From these results the best protection against omega-3 oxidation in these examples was provided by broccoli, mushroom and brussel sprouts.

### Example 17—Oxygen Uptake of Tabletted and Extruded Formulations Using 50% Omega-3 Oil Broccoli Powders

Freeze dried omega-3 oil broccoli powder (50% Hi-DHA tuna oil) prepared as in Example 4 (F1) was used. For the extruded sample, freeze dried omega-3 oil broccoli powder (20% w/w) and maize flour (80% w/w) was dry blended, and feed through an extruder Extruder (DSE32-II Twinscrew Lab Extruder) using a low shear snack extrusion screw profile, barrel temperature of 60° C., 100° C., 140° C., 140° C. (from powder feed to die end) and die pressure: ~5 bar. For the tabletted formulation freeze dried omega-3 oil broccoli powder (50% w/w) and skim milk powder as excipient (50% w/w) was dry blended and tablets (Ø1.3 cm×0.5 cm) made. The oxygen uptake of the samples were assessed by Oxipres test at 80° C. with initial 5 bar oxygen pressure.

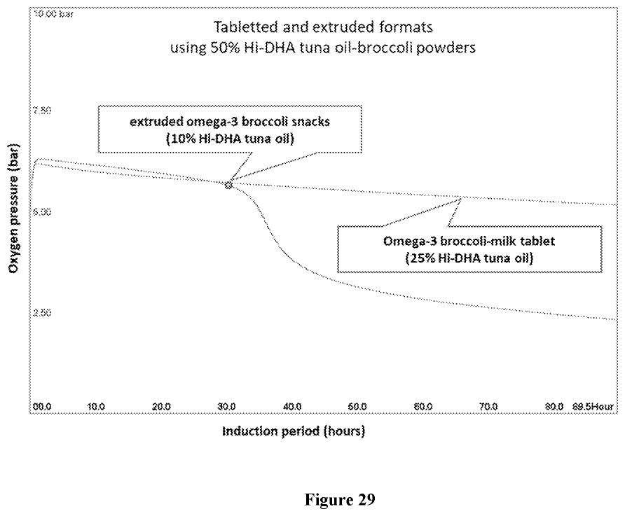

Results are shown in FIG. 29. Sample tested was 40 g for the extrudate (36 g matrix and 4 g oil) and 16 g sample tablet formats (16 g excipient and 4 g oil).

### Example 18—Oxygen Uptake of Omega-3 Oil Broccoli Powders Made by Pre-Treating Broccoli Biomass and/or Post-Treating the Emulsion

Freeze dried broccoli powder was used as encapsulant made up to 5% aqueous solids (hydrated at 50° C., 60 min), and oil was added and homogenised to form a stable emulsion or suspension (9.5% TS and 4.8% oil). Emulsion was freeze dried to make the 50% oil powders. Sample tested contained 4 g oil and 4 g matrix. In this example the biomass (aqueous phase) was either pre-treated using ultrasound (40 KHz/180 W) for 7.5 min in a water bath or microwave (750 W) for 2.5 min to 76.3° C.; or the emulsion was post-treated after homogenisation with high pressure (6000 bar) for 3 min at 25° C. or with microwave (750 W) for 2.5 min to 76.3° C. The oxygen uptake of freeze dried broccoli powders prepared by pre-treating the biomass (5% TS broccoli suspension) or post-treating the emulsion (4.8% oil and 9.5% TS emulsion) was assessed by Oxipres test at 80° C. with initial 5 bar oxygen pressure.

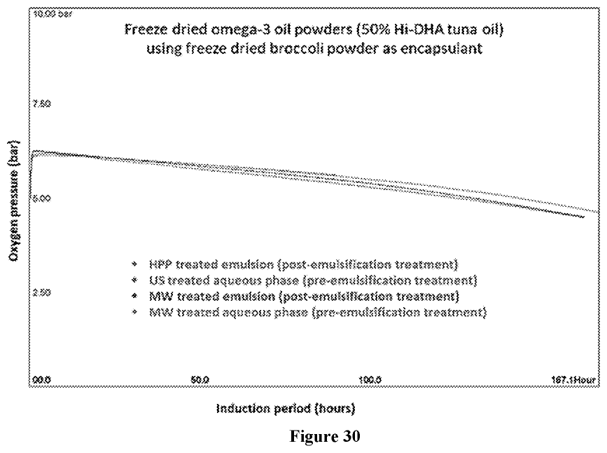

Results are shown in FIG. 30. The slow oxygen uptake in these samples is in part due to the oxygen uptake by the broccoli matrix.

### Example 19—Sulforaphane Content of Broccoli Aqueous Suspensions and Emulsions

Fresh broccoli florets were steamed (core temperature 60° C./5 min) and cooled to room temperature. Aqueous suspensions (5% w/w broccoli solids) and emulsions with medium chain triglycerides (5% w/w broccoli solids and 5% w/w medium chain triglycerides) were prepared and held at 25° C. for 4 hrs. The aqueous suspensions and emulsions were extracted with ethyl acetate and the sulforaphane content measured using UPLC.

There was 13% higher content of sulforaphane (expressed as mg/g dry weight of broccoli solids) in emulsions than in aqueous suspensions. This demonstrates that the sulforaphane was more stable in an emulsion.

### Example 20—SULFORAPHANE CONTENT OF STORED FREEZE DRIED BROCCOLI AQUEOUS SUSPENSIONS AND EMULSIONS

The freeze dried broccoli powder (without oil) and the freeze dried omega-3 oil broccoli powder (50% oil powder) prepared as in Example 6. The sulforaphane content of powders were measured after frozen storage (~18° C. months). Sulforaphane content was measured after extraction of sulforaphane from samples (using an ethyl acetate/hexane mixture). The sulforaphane content (expressed as mg/g dry weight of broccoli solids) in freeze dried omega-3 oil broccoli powder (50% oil) were ~2 fold higher than freeze dried broccoli powder (without oil). This demonstrates that the presence of the oil stabilised the sulforaphane during long term storage.

### Example 21—Discussion

In place of using purified proteins and carbohydrates as encapsulants, these experiments have demonstrated that a whole biomass can be used as an encapsulant, obviating the need for purification and isolation of proteins and carbohydrates from the biomass source. The use of proteins and carbohydrates from the same biomass source can reduce the cost of producing emulsions and suspensions, encapsulants, powders and entrapped and encapsulated bioactive/s and/or bioactive precursor/s. The methods as described herein provide the added advantage of capitalising on all the components inherent in the biomass that have potential to contribute to stabilisation and act as delivery vehicles for bioactive/s and/or bioactive precursor/s. Many biomass sources (e.g. plants, fruits and vegetables, algae, fungi) also contain many nutrients (proteins, carbohydrates, fibres) and phytonutrients (e.g. carotenoids, polyphenols, sulphur-containing compounds, tocopherols, glucosinolates etc) which also have good nutritional and health promoting properties (Hounsome et al., 2008). The ubiquitous presence of proteins, carbohydrates, phytonutrients and other minor components (eg Vitamin C) present in biomass make the use of whole biomass (or fractions of these) instead of purified individual components, and phytonutrients in biomass presents an advantage when using the biomass instead of purified components from biomass for encapsulation and delivery of sensitive bioactive/s and/or bioactive precursor/s. In addition the methods as described herein provide encapsulated oils with a high protection/resistance to oxidative degradation. A comparison of IP for neat oils and oils encapsulated using the methods as described herein is shown in Table 7.

TABLE 7
Comparison of IP for neat oils and encapsulated oil.

| FIG. | | | | | | Relative IP | |
|---|---|---|---|---|---|---|---|
| No. | | | Treatment of | | | (Encapsulated | |
| Reference | Encapsulant | Oil | encapsulant | Oil (%) | IP (hr) | oils: Neat oil) | Comments |
| | OILS (NEAT—WITHOUT ENCAPSULATION) | | | | | | |
| 6 | None | Hi-DHA Tuna oil | n/a | 100 | 9 | | |
| | None | High DHA-Canola oil | n/a | 100 | 11 | | |
| | None | Canola oil | n/a | 100 | 26 | | |
| | TUNA OIL SAMPLES (IP Neat oil 9 hrs) | | | | | | |
| | Emulsions | | | | | | |
| 5a | Tween | Hi-DHA Tuna oil | None | 5 | 9 | 1 | No protection |
| | MicroMAX ® | Hi-DHA Tuna oil | 100° C./30 min | 5 | 11 | 1.2 | |
| | Broccoli (FD) | Hi-DHA Tuna oil | 75° C./2 min | 5 | >20 | >2.2 | Test stopped at 20 hr |
| | Broccoli (FD) | Hi-DHA Tuna oil | 100° C./30 min | 5 | >20 | >2.2 | Test stopped at 20 hr |
| 11 | Broccoli (Raw) | Hi-DHA Tuna oil | 75° C./2 min | 3.8 | >41 | >4.5 | Test stopped at 41 hr |
| | Broccoli (Raw) | Hi-DHA Tuna oil | 100° C./30 min | 5.7 | >41 | >4.5 | Test stopped at 41 hr |
| 14 | Broccoli (Fresh) | Hi-DHA Tuna oil | None | 5 | 10 | 1.1 | |
| | Broccoli (Fresh) | Hi-DHA Tuna oil | Steamed | 5 | 11 | 1.2 | |
| | Broccoli (Fresh) | Hi-DHA Tuna oil | Steamed-shredded | 5 | 12 | 1.3 | |
| | Broccoli (Drum | Hi-DHA Tuna oil | Steamed, Pureed, | 5 | 16 | 1.8 | |
| | dried) | | drum dried | | | | |
| | Freeze dried | | | | | | |
| | Powders | | | | | | |
| 5b | Broccoli (FD) | Hi-DHA Tuna oil | 75° C./2 min | 50 | >42 | >4.6 | Test stopped at 42 hr |
| | Broccoli (FD) | Hi-DHA Tuna oil | 100° C./30 min | 50 | >42 | >4.6 | Test stopped at 42 hr |
| 8 | Broccoli (FD) | Hi-DHA Tuna oil | 79° C./4 min | 12.5 | >334 | >37 | Test stopped at 334 hr |
| 9 | Broccoli (FD) | Hi-DHA Tuna oil | 79° C./4 min | 25 | >305 | >33 | Test stopped at 305 hr |
| 10 | Broccoli (FD) | Hi-DHA Tuna oil | 79° C./4 min | 50 | 104 | 11.5 | |
| | Broccoli (FD) | Hi-DHA Tuna oil | No heat | 25 | 127 | 14.1 | |
| 12 | Broccoli (Raw) | Hi-DHA Tuna oil | 75° C./2 min | 50 | 106 | 11.7 | Made from 9.5% TS emulsion |
| | Broccoli (Raw) | Hi-DHA Tuna oil | 75° C./2 min | 50 | 150 | 16.7 | Made from 11.3% TS emulsion |
| | Broccoli (Raw) | Hi-DHA Tuna oil | 100° C./30 min | 50 | 106 | 11.7 | Made from 9.5% TS emulsion |
| | Broccoli (Raw) | Hi-DHA Tuna oil | 100° C./30 min | 50 | 150 | 16.7 | Made from 11.3% TS emulsion |
| 13 | Broccoli (Fresh) | Hi-DHA Tuna oil | 75° C./2 min | 50 | 106 | 11.7 | Made from 9.5% TS emulsion |
| | | | | | | | (same data in 12) |
| | Broccoli (Fresh) | Hi-DHA Tuna oil | 100° C./30 min | 50 | 106 | 11.7 | Made from 9.5% TS emulsion |
| | | | | | | | (same data in 12) |
| | Broccoli (FD) | Hi-DHA Tuna oil | 75° C./2 min | 50 | 120 | 13.3 | Made from 9.5% TS emulsion |
| | Broccoli (FD) | Hi-DHA Tuna oil | 100° C./30 min | 50 | 120 | 13.3 | Made from 9.5% TS emulsion |
| | Broccoli (Fresh) | Hi-DHA Tuna oil | 75° C./2 min | 50 | 150 | 16.7 | Made from 9.5% TS emulsion |
| | | | | | | | (same data in 12) |
| | Broccoli (Fresh) | Hi-DHA Tuna oil | 100° C./30 min | 50 | 150 | 16.7 | Made from 9.5% TS emulsion |
| | | | | | | | (same data in 12) |
| 15/16 | Carrot (Raw) | Hi-DHA Tuna oil | 75° C./2 min | 50 | >23 | >2.5 | Test stopped at 23 hr, |
| | | | | | | | 8 h over pressure release |
| | Carrot (Raw) | Hi-DHA Tuna oil | 100° C./30 min | 50 | >23 | >2.5 | Test stopped at 23 hr, |
| | | | | | | | 8h over pressure release |
| | Carrot | Hi-DHA Tuna oil | Fermented | 50 | >24 | >2.6 | Test stopped at 24 hr, |
| | | | | | | | 11 h over pressure release |
| 17 | Tomato (Raw) | Hi-DHA Tuna oil | 75° C./2 min | 50 | >23 | >2.5 | Test stopped at 23 hr, |
| | | | | | | | 13 h over pressure release |
| | Tomat0 (Raw) | Hi-DHA Tuna oil | 100° C./30 min | 50 | >23 | >2.5 | Test stopped at 23 hr, |
| | | | | | | | 12 h over pressure release |
| 18 | Mushroom (Raw) | Hi-DHA Tuna oil | 75° C./2 min | 50 | >170 | 18.9 | Test stopped at 170 hr |
| | Mushroom (Raw) | Hi-DHA Tuna oil | 100° C./30 min | 50 | >170 | 18.9 | Test stopped at 170 hr |
| | Mushroom (Raw) | Hi-DHA Tuna oil | 100° C./30 min | 25 | >170 | 18.9 | Test stopped at 170 hr |
| 19 | Cauliflower (Raw) | Hi-DHA Tuna oil | 75° C./2 min | 25 | >88 | >9.8 | Test stopped at 88 hr |
| | Cauliflower (Raw) | Hi-DHA Tuna oil | 75° C./2 min | 50 | 80 | 8.9 | |
| 20 | Kale (Raw) | Hi-DHA Tuna oil | 75° C./2 min | 25 | >144 | >16 | Test stopped at 144 hr |
| | Kale (Raw) | Hi-DHA Tuna oil | 75° C./2 min | 50 | 109 | 12 | |
| 21 | Brussel sprouts | Hi-DHA Tuna oil | 75° C./2 min | 25 | >300 | >33 | Test stopped at 300 hr |
| | (Raw) | | | | | | |
| | Brussel sprouts | Hi-DHA Tuna oil | 75° C./2 min | 50 | 165 | 18 | |
| | (Raw) | | | | | | |
| 22 | Snow peas (Raw) | Hi-DHA Tuna oil | 75° C./2 min | 25 | >163 | >18 | Test stopped at 163 hr |
| | Snow peas (Raw) | Hi-DHA Tuna oil | 75° C./2 min | 50 | >163 | >18 | Test stopped at 163 hr, 71 hr |
| | | | | | | | over pressure release |
| 23 | Garlic (Raw) | Hi-DHA Tuna oil | 75° C./2 min | 25 | 37 | 4.1 | |
| | Garlic (Raw) | Hi-DHA Tuna oil | 75° C./2 min | 50 | >46 | >5.1 | Test stopped at 46 hr, 20 hr |
| | | | | | | | over pressure release |
| 25 | Matcha only | Hi-DHA Tuna oil | — | 25 | >143 | >15.8 | Test stopped at 143 hr |
| | (Dried) | | | | | | |
| | Spray dried | | | | | | |
| | powders | | | | | | |
| 26 | MicoMAX | Hi-DHA Tuna oil | Heated protein- | 50 | 50 | 5.5 | |
| | | | CHO | | | | |
| | Broccoli (ED) | Hi-DHA Tuna oil | 75° C./2 min | 50 | 132 | 14.6 | |
| | DHA CANOLA OIL SAMPLES (IP Neat oil 11 hrs) | | | | | | |
| 9 | Broccoli (ED) | Hi-DHA Canola oil | 79° C./4 min | 25 | >305 | >27.2 | Test stopped at 305 hr |
| 10 | Broccoli (ED) | Hi-DHA Canola oil | No heat | 50 | 95 | 8.6 | |
| | CANOLA OIL SAMPLES (IP Neat oil 26 hrs) | | | | | | |
| 8 | Broccoli (ED) | Canola oil | 79° C./4 min | 12.5 | >334 | >12.8 | Test stopped at 334 hr |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2018900326 entitled “Method of producing an emulsion or suspension and products produced therefrom” filed on 2 Feb. 2018, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Aditya et al. (2017) Biotechnology Advances 35:450-457.
Agerbirk and Olsen (2012) Phytochemisty 77:16-45.
Augustin and Hemar (2009) Chemical Society Reviews 38:902-912.
Augustin et al. (2006) Journal of Food Science 712: E25-E32.
Augustin and Sanguansri (2015) Annual Reviews in Food Science and Technology 6:463-77.
Drusch and Mannino (2009) Trends in Food Science and Technology 20:237-44.
Halkier and Gershenzon (2006) Annual Review in Plant Biology 57:303-33.
Hounsome et al. (2008) Journal of Food Science 3: R48-65.
Hoyos-Leyva et al. (2018) Food Reviews International 34:148-161.
Livney (2010) Current Opinion in Colloid and Interface Science 15:73-83.
McClements (2015) Advances in Colloid and Interface Science 219:27-53.
McCleskey et al. (1947) Journal of Bacteriology 54:697-708.
Olvera C1 et al. (2007) Antonie van Leeuwenhoek 92:11-20.
Sanguansri and Augustin (2006) ed. J. Shi, Taylor & Francis, CRC Press, Boca Raton, FL, USA, 297-327.
Subirade and Chen (2008) ed. N. Garti, CRC Press, New York, USA, 251-278.

1. A method of producing a powder comprising an entrapped or encapsulated bioactive and/or bioactive precursor, the method comprising:
i) obtaining an aqueous mixture comprising protein and carbohydrate from a biomass of a single species of organism;
ii) adding oil to the aqueous mixture;
iii) forming an emulsion or suspension comprising the bioactive and/or bioactive precursor; and
iv) forming a powder comprising an entrapped or encapsulated bioactive and/or bioactive precursor from the emulsion or suspension.

2. The method of claim 1, wherein the powder has an induction period of about 10 to about 300 hours, when measured at 80° C. and a 5 bar initial oxygen pressure.

3. The method of claim 1 or claim 2, wherein the powder comprises about 5% to about 50% oil w/w oil.

4. The method of any one of claims 1 to 3, wherein the powder comprises about 10% to about 40% oil w/w oil.

5. The method of any one of claims 1 to 4, wherein the oil content of emulsion or suspension before forming the powder is from about 1% to about 10% w/w.

6. The method of any one of claims 1 to 5, wherein the aqueous mixture further comprises protein and carbohydrate from at least one further biomass from a single species of organism.

7. The method of any one of claims 1 to 6, wherein the biomass and/or further biomass comprises one or more of:
i) a protein to carbohydrate ratio of between about 1:1 to 1:10.5;
ii) a protein to carbohydrate ratio of between about 1:4.5 and about 4:1; and
iii) a protein to carbohydrate ratio of between about 1:2.5 and about 2:1.

8. The method of any one of claims 1 to 7, wherein the bioactive and/or bioactive precursor is one or more of:
i) a component of the biomass;
ii) the oil or a component thereof in step ii);
iii) a component added to the oil before the oil is added to the aqueous mixture in step ii);
iv) a component infused in the oil before or during step ii);
v) a component of the further biomass; and
vi) is a component added in step i), ii) and iii) of the method.

9. The method of claim 8, wherein the bioactive is i) and ii).

10. The method of claim 8, wherein the bioactive precursor is i).

11. The method of claim 8, wherein the bioactive is formed in or after step i), ii) or iii).

12. The method of any one of claims 1 to 11, wherein the bioactive and/or bioactive precursor is sensitive to one or more of: degradation by oxygen, temperature, pH, moisture and light.

13. The method of any one of claims 1 to 12, wherein when the biomass and/or further biomass comprises:
i) Brassicaceae and the bioactive is an isothiocyanate;
ii) Brassicaceae and the bioactive precursor is a glucosinolate and/or glucoraphanin;
iii) onion and the bioactive is one or more of quercetin, allicin and phenolic acid;
iv) garlic and the bioactive is one or more of allicin and ajoene; or
v) fruit and/or vegetables containing polyphenols.

14. The method of any one of claims 1 to 13, wherein the bioactive is a phytonutrient.

15. The method of any one of claims 1 to 14, wherein bioactive is selected from one or more of: fatty acid, an isothiocyanate, quercetin, allicin, ajoene, vitamin A, vitamin D, vitamin E, tocopherols, tocotrienols, vitamin K, beta-carotene, lycopene, lutein, zeaxanthin, stigmasterol, beta-sitosterol, campesterol, antioxidants, coenzyme Q10, astaxanthin, cannabinoid, cannabiodiol and a polyphenol.

16. The method of claim 15, wherein the fatty acid is: omega-3, omega-6 or omega-9 fatty acid.

17. The method of claim 16, wherein the omega-3 fatty acid is one or more of a-linolenic acid (ALA), eicosapentaenoic acid (EPA), docosapentaenoic acid (DPA) and docosahexaenoic acid (DHA).

18. The method of any one of claims 1 to 17, further comprising pre-treating the biomass.

19. The method of claim 18, wherein pre-treating comprises one or more of:
i) heating;
ii) macerating;
iii) microwaving;
iv) exposure to low frequency sound waves (ultrasound);
v) pulse electric field processing;
vi) static high pressure;
vii) extrusion;
viii) enzyme treatment;
ix) fermentation;
x) an extraction or separation process; and
xi) drying.

20. The method of any one of claims 1 to 19, wherein the biomass or further biomass is from the Plantae or Fungi Kingdom.

21. The method of claim 20, wherein the Plantae is selected from: Brassicaceae, Cannabis Asparagaceae, Arecaceae, Myrtaceae, Rosaceae, Musaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae, Caesalpinioideae, Compositae, Amaranthaceae/Chenopodiacae, Malvaceae, Amarylidaceae, Fabaceae, Arecaceae and Poaceae.

22. The method of claim 20, where the Plantae is selected from: fruit, vegetable, nut, legume, cereal and a grass.

23. The method of claim 22, wherein the Plantae is selected from: broccoli, kale, cauliflower, carrot, onion, garlic, tomato, brussel sprouts, spinach, snow peas, asparagus, and avocado.

24. The method of claim 20, wherein the Fungi is selected from: Boletaceae, Cantharellaceae, Tricholomataceae, Cortinariaceae, Cantharellaceae, Meripilaceae, Discinaceae, Pleurotaceae, Tricholomataceae and Tuberaceae.

25. The method of any one of claims 1 to 20, wherein the biomass or further biomass is matcha, canola meal, nut meal, soybean meal, coconut meal, palm kernel meal, hemp oil press cakes, chia oil seed cake or rice bran.

26. The method of any one of claims 1 to 25, wherein the oil comprises one or more fatty acids.

27. The method of any one of claims 1 to 26, wherein the oil is selected from one or more of: fish oil, krill oil, marine oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, medium chain triglycerides and grapeseed oil.

28. The method of claim 27, wherein the fish oil or marine oil is selected from one or more of: tuna oil, herring oil, mackerel oil, sardine oil, cod liver oil, menhaden oil, shark oil, algal oil, squid oil, and squid liver oil.

29. The method of any one of claims 1 to 28, wherein the method comprises post-treating the emulsion or suspension to reduce microbial activity.

30. The method of claim 29, wherein post-treating comprises one or more of the following:
i) heating;
ii) microwaving;
iii) UV treatment; and
iv) high pressure processing.

31. The method of any one of claims 1 to 30, wherein forming the powder comprises spray drying, freeze drying, refractance window drying or drum drying.

32. The method of any one of claims 1 to 31, wherein when the entrapped or encapsulated bioactive and/or bioactive precursor is resistant to degradation for about 3 to about 24 months.

33. The method of claim 32, wherein the degradation is selected from one or more of: oxygen, temperature, pH, moisture and light.

34. The method of any one of claims 1 to 33, wherein the powder is extruded.

35. The method of claim 34, wherein the powder is extruded or compressed to form a tablet.

36. A powder comprising an entrapped or encapsulated bioactive and/or bioactive precursor and comprising protein and carbohydrate from a single species of organism.

37. The powder of claim 36, produced by the method of any one of claims 1 to 35.

38. The powder of claim 36 or claim 37, wherein the entrapped or encapsulated bioactive and/or bioactive precursor is resistant to oxygen degradation compared to the unentrapped or unencapsulated bioactive and/or bioactive precursor.

39. The powder of any one of claims 36 to 38, wherein the bioactive and/or bioactive precursor is a fatty acid.

40. The powder of any one of claims 36 to 39, wherein the powder has an induction period of about 10 to about 300 hours, when measured at 80° C. and a 5 bar initial oxygen pressure.

41. The powder of any one of claims 36 to 40, wherein the emulsion or suspension has an induction period of at least 100 hours, when measured at 80° C. and a 5 bar initial oxygen pressure.

42. The powder of any one of claims 36 to 41, wherein the powder comprises about 5% to about 50% oil w/w oil.

43. The powder of any one of claims 36 to 42, wherein the powder comprises about 10% to about 40% oil w/w oil.

44. The powder of any one of claims 36 to 43, wherein the bioactive and/or bioactive precursor is one or more of:
i) a component of the biomass;
ii) the oil or a component thereof in step ii);
iii) a component added to the oil before the oil is added to the aqueous mixture in step ii);
iv) a component infused in the oil before or during step ii);
v) a component of the further biomass; and
vi) is a component added in step i), ii) and iii) of the method.

45. The powder of claim 44, wherein the bioactive is i) and ii).

46. A product comprising the powder produced by the method of any one of claims 1 to 35, or the powder of any one of claims 36 to 45.

47. The product of claim 46, wherein the entrapped or encapsulated bioactive and/or bioactive precursor in the product is more resistant to degradation compared to the same product comprising unentrapped or unencapsulated bioactive and/or bioactive precursor.

48. The product of claim 46 or 47, wherein the product is a cream, gel tablet, liquid, pill, capsule, powder or extruded product.

49. The product of any one of claims 46 to 48, wherein the product is a food, food ingredient, supplement, cosmetic or cosmetic ingredient.

50. The product of any one of claims 46 to 49, wherein the product comprises omega-3 polyunsaturated fatty acids.

51. The product of any one of claims 46 to 50, wherein the food is an animal feed.

52. The product of claim 51, wherein the animal feed is an aquaculture feed.

53. A method of producing an emulsion or suspension, the method comprising:
i) obtaining an aqueous suspension comprising protein and carbohydrate from a biomass of a single species of organism;
ii) optionally adding oil to the aqueous suspension; and
iii) forming an emulsion or suspension comprising a bioactive and/or bioactive precursor.

54. A matrix comprising protein and carbohydrate from a biomass of a single species of organism.

55. A bioactive and/or bioactive precursor entrapped or encapsulated in a matrix comprising protein and carbohydrate of a biomass from a single species of organism, wherein the entrapped or encapsulated bioactive and/or bioactive precursor is resistant to oxygen degradation when compared to the bioactive and/or bioactive precursor before entrapment or encapsulation.

56. An emulsion or suspension produced by the method of claim 53.

57. A product comprising the emulsion or suspension produced by the method of claim 53, the matrix of claim 54, the bioactive and/or bioactive precursor entrapped or encapsulated in the matrix of claim 55, or the emulsion or suspension of claim 56.

58. A pharmaceutical or cosmetic composition, which comprises an isothiocyanate and/or an isothiocyanate precursor, a lipid, and a pharmaceutical and/or cosmetic excipient.

59. The composition of claim 58, wherein the composition is for topical, enteral/gastrointestinal or parenteral administration.

60. The composition of claim 58 or claim 59, wherein the composition is in the form of a cream, ointment, gel, tablet, liquid, pill, capsule, powder or extruded product.

61. The composition of any one of claims 58 to 60, wherein following storage for a period of about one month, at least 50% of the isothiocyanate and/or isothiocyanate precursor remain in the composition.

62. A method of producing an emulsion comprising an isothiocyanate or isothiocyanate precursor, the method comprising:
providing a mixture comprising water, a lipid, and an isothiocyanate or isothiocyanate precursor, thereby forming an emulsion.

63. The method of claim 62, wherein following about one month of storage at about 4 to about 10° C. or at about −18° C., the concentration of the isothiocyanate and/or isothiocyanate precursor in the emulsion is at least twice the concentration of isothiocyanate and/or isothiocyanate precursor in a corresponding composition lacking the lipid.

64. An emulsion comprising water, a lipid, and an isothiocyanate and/or isothiocyanate precursor.

65. A method of preparing a powder comprising an isothiocyanate and/or isothiocyanate precursor, comprising:
preparing an emulsion according to any one of claims 62 to 64, and
subjecting the emulsion to drying conditions, thereby removing water and forming a powder.

66. The method of claim 65, wherein the emulsion is subjected to freeze-drying, spray drying conditions or refractance window drying thereby forming a powder.

67. The method of claim 65 or claim 66, wherein following about two months of storage at −18° C., the concentration of the isothiocyanate and/or isothiocyanate precursor in the powder is at least twice the concentration of isothiocyanate and/or isothiocyanate precursor in a corresponding powder lacking the lipid.

68. A method of preparing a pharmaceutical or cosmetic composition, comprising:
preparing an emulsion of any one of claims 62 to 63, or preparing a powder of claim 65 or claim 67, and
converting the emulsion or dried composition to a pharmaceutical or cosmetic composition.

69. The composition, method, or emulsion of any one of claims 58 to 68, wherein the isothiocyanate is selected from one or more of: sulforaphane, allyl isothiocyanate, benzyl isothiocyanate and phenethyl isothiocyanate.

70. The composition, method, or emulsion of any one of claims 58 to 69, wherein the isothiocyanate precursor is selected from one or more of: glucosinolate, glucoraphanin, sinigrin, glucotropaeolin, and gluconasturtiin.

71. The composition, method, or emulsion of any one of claims 58 to 70, wherein the lipid is an oil.

72. A method of therapy or prophylaxis of a condition, comprising administering to a subject in need thereof an effective amount of a pharmaceutical composition, emulsion or powder of any one of claims 58 to 71.

73. A pharmaceutical composition, emulsion or powder of any one of claims 58 to 72, for use in therapy or prophylaxis of a condition.

74. A method of treating or preventing a condition in a subject, comprising administering to the subject an effective amount of a pharmaceutical composition, emulsion or powder of any one of claims 58 to 73.

75. Use of the emulsion of claim 64, or the powder of claim 65 or 66 in the manufacture of a medicament for the treatment of a condition.

76. The method or use of any one of claim 72, 73, 74 or 75, wherein the condition is selected from: cancer, diabetes, cardiovascular disease, autism, osteoporosis, neuroprotective diseases, metabolic syndrome, inflammation, oxidative stress and a gut health condition.
